SEQUENCE LISTING (48 sequences)

SEQ ID NO: 1 - DNA, 8684 bp, Escherichia coli
  gene (63)..(866): strA
aactgcacattcgggatatttctctatattcgcgcttcatcagaaaactgaaggaacctccattgaatcgaactaatattttttttggtgaatcgcattctgactggttgcctgtcagaggcggagaatctggtgattttgtttttcgacgtggtgacgggcatgccttcgcgaaaatcgcacctgcttcccgccgcggtgagctcgctggagagcgtgaccgcctcatttggctcaaaggtcgaggtgtggcttgccccgaggtcatcaactggcaggaggaacaggagggtgcatgcttggtgataacggcaattccgggagtaccggcggctgatctgtctggagcggatttgctcaaagcgtggccgtcaatggggcagcaacttggcgctgttcacagcctatcggttgatcaatgtccgtttgagcgcaggctgtcgcgaatgttcggacgcgccgttgatgtggtgtcccgcaatgccgtcaatcccgacttcttaccggacgaggacaagagtacgccgctgcacgatcttttggctcgtgtcgaacgagagctaccggtgcggctcgaccaagagcgcaccgatatggttgtttgccatggtgatccctgcatgccgaacttcatggtggaccctaaaactcttcaatgcacgggtctgatcgaccttgggcggctcggaacagcagatcgctatgccgatttggcactcatgattgctaacgccgaagagaactgggcagcgccagatgaagcagagcgcgccttcgctgtcctattcaatgtattggggatcgaagcccccgaccgcgaacgccttgccttctatctgcgattggaccctctgacttggggttgatgttcatgccgcctgtttttcctgctcattggcacgtttcgcaacctgttctcattgcggacaccttttccagcctcgtttggaaagtttcattgccagacgggactcctgcaatcgtcaagggattgaaacctatagaagacattgctgatgaactgcgcggggccgactatctggtatggcgcaatgggaggggagcagtccggttgctcggtcgtgagaacaatctgatgttgctcgaatatgccggggagcgaatgctctctcacatcgttgccgagcacggcgactaccaggcgaccgaaattgcagcggaactaatggcgaagctgtatgccgcatctgaggaacccctgccttctgcccttctcccgatccgggatcgctttgcagctttgtttcagcgggcgcgcgatgatcaaaacgcaggttgtcaaactgactacgtccacgcggcgattatagccgatcaaatgatgagcaatgcctcggaactgcgtgggctacatggcgatctgcatcatgaaaacatcatgttctccagtcgcggctggctggtgatagatcccgtcggtctggtcggtgaagtgggctttggcgccgccaatatgttctacgatccggctgacagagacgacctttgtctcgatcctagacgcattgcacagatggcggacgcattctctcgtgcgctggacgtcgatccgcgtcgcctgctcgaccaggcgtacgcttatgggtgcctttccgcagcttggaacgcggatggagaagaggagcaacgcgatctagctatcgcggccgcgatcaagcaggtgcgacagacgtcatactagatatcaagcgacttctcctatcccctgggaacacatcaatctcaccggagaatatcgctggccaaagccttagcgtaggattccgccccttcccgcaaacgaccccaaacaggaaacgcagctgaaacgggaagctcaacacccactgacgcatgggttgttcaggcagtacttcatcaaccagcaaggcggcactttcggccatccgccgcgccccacagctcgggcagaaaccgcgacgcttacagctgaaagcgaccaggtgctcggcgtggcaagactcgcagcgaacccgtagaaagccatgctccagccgcccgcattggagaaattcttcaaattcccgttgcacatagcccggcaattcctttccctgctctgccataagcgcagcgaatgccgggtaatactcgtcaacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagcaacagcgaggcagcatggcgatttatcaccttacggcgaaaaccggcagcaggtcgggcggccaatcggccagggccaaggccgactacatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagctaatggatcaccgcaaacaggttactcgcctggggattccctttcgacccgagcatccgtatgatactcatgctcgattattattattatagaagcccccatgaataaatcgctcatcattttcggcatcgtcaacataacctcggacagtttctccgatggaggccggtatctggcgccagacgcagccattgcgcaggcgcgtaagctgatggccgagggggcagatgtgatcgacctggtccggcatccagcaatcccgacgccgcgcctgtttcgtccgacacagaaatcgcgcgtatgcgccggtgctggacgcgctcaggcagatggcattcccgtctcgctcgacagttatcaacccgcgacgcaagcctatgccttgtcgcgtggtgtggcctatctcaatgatattcgcggttttccagacgctgcgttctatccgcaattggcgaaatcatctgccaaactcgtcgttatgcattcggtgcaagacgggcaggcagatcggcgcgaggcacccgctggcgacatcatggatcacattgcggcgttctttgacgcgcgcatcgcggcgctgacgggtgccggtatcaaacgcaaccgccttgtccttgatcccggcatggggttttttctgggggctgctcccgaaacctcgctctcggtgctggcgcggttcgatgaattgcggctgcgcttcgatttgccggtgcttctgtctgtttcgcgcaaatcctttctgcgcgcgctcacaggccgtggtccgggggtgtcggggccgcgacactcgctgcagagcttgccgccgccgcaggtggagctgacttcatccgcacacacgagccgcgccccttgcgcgacgggctggcggtattggcggcgctgaaagaaaccgcaagaattcgtt

SEQ ID NO: 2 - DNA, 804 bp, Escherichia coli
  CDS (1)..(804): strA
ttgaatcgaactaatattttttttggtgaatcgcattctgactggttgcctgtcagaggcggagaatctggtgattttgtttttcgacgtggtgacgggcatgccttcgcgaaaatcgcacctgcttcccgccgcggtgagctcgctggagagcgtgaccgcctcatttggctcaaaggtcgaggtgtggcttgccccgaggtcatcaactggcaggaggaacaggagggtgcatgcttggtgataacggcaattccgggagtaccggcggctgatctgtctggagcggatttgctcaaagcgtggccgtcaatggggcagcaacttggcgctgttcacagcctatcggttgatcaatgtccgtttgagcgcaggctgtcgcgaatgttcggacgcgccgttgatgtggtgtcccgcaatgccgtcaatcccgacttcttaccggacgaggacaagagtacgccgctgcacgatcttttggctcgtgtcgaacgagagctaccggtgcggctcgaccaagagcgcaccgatatggttgtttgccatggtgatccctgcatgccgaacttcatggtggaccctaaaactcttcaatgcacgggtctgatcgaccttgggcggctcggaacagcagatcgctatgccgatttggcactcatgattgctaacgccgaagagaactgggcagcgccagatgaagcagagcgcgccttcgctgtcctattcaatgtattggggatcgaagcccccgaccgcgaacgccttgccttctatctgcgattggaccctctgacttggggttga

SEQ ID NO: 3 - protein, 267 aa, Escherichia coli
LNRTNIFFGESHSDWLPVRGGESGDFVFRRGDGHAFAKIAPASRRGELAGERDRLIWLKGRGVACPEVINWQEEQEGACLVITAIPGVPAADLSGADLLKAWPSMGQQLGAVHSLSVDQCPFERRLSRMFGRAVDVVSRNAVNPDFLPDEDKSTPLHDLLARVERELPVRLDQERTDMVVCHGDPCMPNFMVDPKTLQCTGLIDLGRLGTADRYADLALMIANAEENWAAPDEAERAFAVLFNVLGIEAPDRERLAFYLRLDPLTWG

SEQ ID NO: 4 - DNA, 837 bp, Escherichia coli
  CDS (1)..(837): strB
atgttcatgccgcctgtttttcctgctcattggcacgtttcgcaacctgttctcattgcggacaccttttccagcctcgtttggaaagtttcattgccagacgggactcctgcaatcgtcaagggattgaaacctatagaagacattgctgatgaactgcgcggggccgactatctggtatggcgcaatgggaggggagcagtccggttgctcggtcgtgagaacaatctgatgttgctcgaatatgccggggagcgaatgctctctcacatcgttgccgagcacggcgactaccaggcgaccgaaattgcagcggaactaatggcgaagctgtatgccgcatctgaggaacccctgccttctgcccttctcccgatccgggatcgctttgcagctttgtttcagcgggcgcgcgatgatcaaaacgcaggttgtcaaactgactacgtccacgcggcgattatagccgatcaaatgatgagcaatgcctcggaactgcgtgggctacatggcgatctgcatcatgaaaacatcatgttctccagtcgcggctggctggtgatagatcccgtcggtctggtcggtgaagtgggctttggcgccgccaatatgttctacgatccggctgacagagacgacctttgtctcgatcctagacgcattgcacagatggcggacgcattctctcgtgcgctggacgtcgatccgcgtcgcctgctcgaccaggcgtacgcttatgggtgcctttccgcagcttggaacgcggatggagaagaggagcaacgcgatctagctatcgcggccgcgatcaagcaggtgcgacagacgtcatactag

SEQ ID NO: 5 - protein, 278 aa, Escherichia coli
MFMPPVFPAHWHVSQPVLIADTFSSLVWKVSLPDGTPAIVKGLKPIEDIADELRGADYLVWRNGRGAVRLLGRENNLMLLEYAGERMLSHIVAEHGDYQATEIAAELMAKLYAASEEPLPSALLPIRDRFAALFQRARDDQNAGCQTDYVHAAIIADQMMSNASELRGLHGDLHHENIMFSSRGWLVIDPVGLVGEVGFGAANMFYDPADRDDLCLDPRRIAQMADAFSRALDVDPRRLLDQAYAYGCLSAAWNADGEEEQRDLAIAAAIKQVRQTSY

SEQ ID NO: 6 - DNA, 285 bp, Escherichia coli
  CDS (1)..(285): mobC
atggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctga

SEQ ID NO: 7 - protein, 94 aa, Escherichia coli
MVKGSNKAADRLAKLEEQRARINAEIQRVRAREQQQERKNETRRKVLVGAMILAKVNSSEWPEDRLMAAMDAYLERDHDRALFGLPPRQKDEPG

SEQ ID NO: 8 - DNA, 2130 bp, Escherichia coli
  CDS (1)..(2130): mobA
atggcgatttatcaccttacggcgaaaaccggcagcaggtcgggcggccaatcggccagggccaaggccgactacatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtag

SEQ ID NO: 9 - protein, 709 aa, Escherichia coli
MAIYHLTAKTGSRSGGQSARAKADYIQREGKYARDMDEVLHAESGHMPEFVERPADYWDAADLYERANGRLFKEVEFALPVELTLDQQKALASEFAQHLTGAERLPYTLAIHAGGGENPHCHLMISERINDGIERPAAQWFKRYNGKTPEKGGAQKTEALKPKAWLEQTREAWADHANRALERAGHDARIDHRTLEAQGIERLPGVHLGPNVVEMEGRGIRTDRADVALNIDTANAQIIDLQEYREAIDHERNRQSEEIQRHQRVSGADRTAGPEHGDTGRRSPAGHEPDPAGQRGAGGGVAESPAPDRGGMGGAGQRVAGGSRRGEQRRAERPERVAGVALEAMANRDAGFHDAYGGAADRIVALARPDATDNRGRLDLAALGGPMKNDRTLQAIGRQLKAMGCERFDIGVRDATTGQMMNREWSAAEVLQNTPWLKRMNAQGNDVYIRPAEQERHGLVLVDDLSEFDLDDMKAEGREPALVVETSPKNYQAWVKVADAAGGELRGQIARTLASEYDADPASADSRHYGRLAGFTNRKDKHTTRAGYQPWVLLRESKGKTATAGPALVQQAGQQIEQAQRQQEKARRLASLELPERQLSRHRRTALDEYRSEMAGLVKRFGDDLSKCDFIAAQKLASRGRSAEEIGKAMAEASPALAERKPGHEADYIERTVSKVMGLPSVQLARAELARAPAPRQRGMDRGGPDFSM

SEQ ID NO: 10 - DNA, 414 bp, Escherichia coli
  CDS (1)..(414): mobB
atgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatga

SEQ ID NO: 11 - protein, 137 aa, Escherichia coli
MNAIDRVKKSRGINELAEQIEPLAQSMATLADEARQVMSQTQQASEAQAAEWLKAQRQTGAAWVELAKELREVAAEVSSAAQSARSASRGWHWKLWLTVMLASMMPTVVLLIASLLLLDLTPLTTEDGSIWLRLVAR

SEQ ID NO: 12 - DNA, 972 bp, Escherichia coli
  CDS (1)..(972): repB
atgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtag

SEQ ID NO: 13 - protein, 323 aa, Escherichia coli
MKNDRTLQAIGRQLKAMGCERFDIGVRDATTGQMMNREWSAAEVLQNTPWLKRMNAQGNDVYIRPAEQERHGLVLVDDLSEFDLDDMKAEGREPALVVETSPKNYQAWVKVADAAGGELRGQIARTLASEYDADPASADSRHYGRLAGFTNRKDKHTTRAGYQPWVLLRESKGKTATAGPALVQQAGQQIEQAQRQQEKARRLASLELPERQLSRHRRTALDEYRSEMAGLVKRFGDDLSKCDFIAAQKLASRGRSAEEIGKAMAEASPALAERKPGHEADYIERTVSKVMGLPSVQLARAELARAPAPRQRGMDRGGPDFSM

SEQ ID NO: 14 - DNA, 213 bp, Escherichia coli
  CDS (1)..(213): orfE
atggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactga

SEQ ID NO: 15 - protein, 70 aa, Escherichia coli
MEYEKSASGSVYLIKSDKGYWLPGGFGYTSNKAEAGRFSVADMASLNLDGCTLSLFREDKPFGPGKFLGD

SEQ ID NO: 16 - DNA, 207 bp, Escherichia coli
  CDS (1)..(207): orfF
atgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaa

SEQ ID NO: 17 - protein, 68 aa, Escherichia coli
MKDQKDKQTGDLLASPDAVRQARYAERMKAKGMRQRKFWLTDDEYEALRECLEELRAAQGGGSDPASA

SEQ ID NO: 18 - DNA, 840 bp, Escherichia coli
  CDS (1)..(840): repA
atggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaa

SEQ ID NO: 19 - protein, 279 aa, Escherichia coli
MATHKPINILEAFAAAPPPLDYVLPNMVAGTVGALVSPGGAGKSMLALQLAAQIAGGPDLLEVGELPTGPVIYLPAEDPPTAIHHRLHALGAHLSAEERQAVADGLLIQPLIGSLPNIMAPEWFDGLKRAAEGRRLMVLDTLRRFHIEEENASGPMAQVIGRMEAIAADTGCSIVFLHHASKGAAMMGAGDQQQASRGSSVLVDNIRWQSYLSSMTSAEAEEWGVDDDQRRFFVRFGVSKANYGAPFADRWFRRHDGGVLKPAVLERQRKSKGVPRGEA

SEQ ID NO: 20 - DNA, 852 bp, Escherichia coli
  CDS (1)..(852): repC
gtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctga

SEQ ID NO: 21 - protein, 283 aa, Escherichia coli
VVKPKNKHSLSHVRHDPAHCLAPGLFRALKRGERKRSKLDVTYDYGDGKRIEFSGPEPLGADDLRILQGLVAMAGPNGLVLGPEPKTEGGRQLRLFLEPKWEAVTAECHVVKGSYRALAKEIGAEVDSGGALKHIQDCIERLWKVSIIAQNGRKRQGFRLLSEYASDEADGRLYVALNPLIAQAVMGGGQHVRISMDEVRALDSETARLLHQRLCGWIDPGKTGKASIDTLCGYVWPSEASGSTMRKRRQRVREALPELVALGWTVTEFAAGKYDITRPKAAG

SEQ ID NO: 22 - DNA, 789 bp, Escherichia coli
  CDS (1)..(789): sul
atgaataaatcgctcatcattttcggcatcgtcaacataacctcggacagtttctccgatggaggccggtatctggcgccagacgcagccattgcgcaggcgcgtaagctgatggccgagggggcagatgtgatcgacctggtccggcatccagcaatcccgacgccgcgcctgtttcgtccgacacagaaatcgcgcgtatgcgccggtgctggacgcgctcaggcagatggcattcccgtctcgctcgacagttatcaacccgcgacgcaagcctatgccttgtcgcgtggtgtggcctatctcaatgatattcgcggttttccagacgctgcgttctatccgcaattggcgaaatcatctgccaaactcgtcgttatgcattcggtgcaagacgggcaggcagatcggcgcgaggcacccgctggcgacatcatggatcacattgcggcgttctttgacgcgcgcatcgcggcgctgacgggtgccggtatcaaacgcaaccgccttgtccttgatcccggcatggggttttttctgggggctgctcccgaaacctcgctctcggtgctggcgcggttcgatgaattgcggctgcgcttcgatttgccggtgcttctgtctgtttcgcgcaaatcctttctgcgcgcgctcacaggccgtggtccgggggtgtcggggccgcgacactcgctgcagagcttgccgccgccgcaggtggagctgacttcatccgcacacacgagccgcgccccttgcgcgacgggctggcggtattggcggcgctga

SEQ ID NO: 23 - protein, 262 aa, Escherichia coli
MNKSLIIFGIVNITSDSFSDGGRYLAPDAAIAQARKLMAEGADVIDLVRHPAIPTPRLFRPTQKSRVCAGAGRAQADGIPVSLDSYQPATQAYALSRGVAYLNDIRGFPDAAFYPQLAKSSAKLVVMHSVQDGQADRREAPAGDIMDHIAAFFDARIAALTGAGIKRNRLVLDPGMGFFLGAAPETSLSVLARFDELRLRFDLPVLLSVSRKSFLRALTGRGPGVSGPRHSLQSLPPPQVELTSSAHTSRAPCATGWRYWRR

SEQ ID NO: 24 - DNA, 8335 bp, Escherichia coli
  gene (63)..(866): strA
aactgcacattcgggatatttctctatattcgcgcttcatcagaaaactgaaggaacctccattgaatcgaactaatattttttttggtgaatcgcattctgactggttgcctgtcagaggcggagaatctggtgattttgtttttcgacgtggtgacgggcatgccttcgcgaaaatcgcacctgcttcccgccgcggtgagctcgctggagagcgtgaccgcctcatttggctcaaaggtcgaggtgtggcttgccccgaggtcatcaactggcaggaggaacaggagggtgcatgcttggtgataacggcaattccgggagtaccggcggctgatctgtctggagcggatttgctcaaagcgtggccgtcaatggggcagcaacttggcgctgttcacagcctatcggttgatcaatgtccgtttgagcgcaggctgtcgcgaatgttcggacgcgccgttgatgtggtgtcccgcaatgccgtcaatcccgacttcttaccggacgaggacaagagtacgccgctgcacgatcttttggctcgtgtcgaacgagagctaccggtgcggctcgaccaagagcgcaccgatatggttgtttgccatggtgatccctgcatgccgaacttcatggtggaccctaaaactcttcaatgcacgggtctgatcgaccttgggcggctcggaacagcagatcgctatgccgatttggcactcatgattgctaacgccgaagagaactgggcagcgccagatgaagcagagcgcgccttcgctgtcctattcaatgtattggggatcgaagcccccgaccgcgaacgccttgccttctatctgcgattggaccctctgacttggggttgatgttcatgccgcctgtttttcctgctcattggcacgtttcgcaacctgttctcattgcggacaccttttccagcctcgtttggaaagtttcattgccagacgggactcctgcaatcgtcaagggattgaaacctatagaagacattgctgatgaactgcgcggggccgactatctggtatggcgcaatgggaggggagcagtccggttgctcggtcgtgagaacaatctgatgttgctcgaatatgccggggagcgaatgctctctcacatcgttgccgagcacggcgactaccaggcgaccgaaattgcagcggaactaatggcgaagctgtatgccgcatctgaggaacccctgccttctgcccttctcccgatccgggatcgctttgcagctttgtttcagcgggcgcgcgatgatcaaaacgcaggttgtcaaactgactacgtccacgcggcgattatagccgatcaaatgatgagcaatgcctcggaactgcgtgggctacatggcgatctgcatcatgaaaacatcatgttctccagtcgcggctggctggtgatagatcccgtcggtctggtcggtgaagtgggctttggcgccgccaatatgttctacgatccggctgacagagacgacctttgtctcgatcctagacgcattgcacagatggcggacgcattctctcgtgcgctggacgtcgatccgcgtcgcctgctcgaccaggcgtacgcttatgggtgcctttccgcagcttggaacgcggatggagaagaggagcaacgcgatctagctatcgcggccgcgatcaagcaggtgcgacagacgtcatactagatatcaagcgacttctcctatcccctgggaacacatcaatctcaccggagaatatcgctggccaaagccttagcgtaggattccgccccttcccgcaaacgaccccaaacaggaaacgcagctgaaacgggaagctcaacacccactgacgcatgggttgttcaggcagtacttcatcaaccagcaaggcggcactttcggccatccgccgcgccccacagctcgggcagaaaccgcgacgcttacagctgaaagcgaccaggtgctcggcgtggcaagactcgcagcgaacccgtagaaagccatgctccagccgcccgcattggagaaattcttcaaattcccgttgcacatagcccggcaattcctttccctgctctgccataagcgcagcgaatgccgggtaatactcgtcaacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatagatctgcgggcagtgagcgcaacgcaattaatgtgagttagctcactcattaggcaccccaggctttacactttatgcttccggctcgtataatgtgtggaattgtgagcggataacaatttcacacaggatctagaaataattttgtttaactttaagaaggagatatacatatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaaccgtcgaagcctgtaaagcggcggtgcacaatcttctcgcgcaacgcgtcagtgggctgatagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaggatccggggggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagctaatggatcaccgcaaacaggttactcgcctggggattccctttcgacccgagcatccgtatgatactcatgctcgattattattattatagaagcccccatgaataaatcgctcatcattttcggcatcgtcaacataacctcggacagtttctccgatggaggccggtatctggcgccagacgcagccattgcgcaggcgcgtaagctgatggccgagggggcagatgtgatcgacctggtccggcatccagcaatcccgacgccgcgcctgtttcgtccgacacagaaatcgcgcgtatgcgccggtgctggacgcgctcaggcagatggcattcccgtctcgctcgacagttatcaacccgcgacgcaagcctatgccttgtcgcgtggtgtggcctatctcaatgatattcgcggttttccagacgctgcgttctatccgcaattggcgaaatcatctgccaaactcgtcgttatgcattcggtgcaagacgggcaggcagatcggcgcgaggcacccgctggcgacatcatggatcacattgcggcgttctttgacgcgcgcatcgcggcgctgacgggtgccggtatcaaacgcaaccgccttgtccttgatcccggcatggggttttttctgggggctgctcccgaaacctcgctctcggtgctggcgcggttcgatgaattgcggctgcgcttcgatttgccggtgcttctgtctgtttcgcgcaaatcctttctgcgcgcgctcacaggccgtggtccgggggtgtcggggccgcgacactcgctgcagagcttgccgccgccgcaggtggagctgacttcatccgcacacacgagccgcgccccttgcgcgacgggctggcggtattggcggcgctgaaagaaaccgcaagaattcgtt

SEQ ID NO: 25 - DNA, 1083 bp, Escherichia coli
  CDS (1)..(1083): lacI
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 26 - protein, 360 aa, Escherichia coli
VKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 27 - DNA, 6864 bp, Escherichia coli
  gene (196)..(990): thyA
gaattcctgattggttacggcgcgtttcgcatcattgttgagtttttccgccagcccgacgcgcagtttaccggtgcctgggtgcatgatataatcatggggcaaattctttccatcccgatgattgtcgcgggtgtgatcatgatggtctgggcatatcgtcgcagcccacagcaacacgtttcctgaggaaccatgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttctccagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaattacgaagcttgcggccgcgatcaagcaggtgcgacagacgtcatactagatatcaagcgacttctcctatcccctgggaacacatcaatctcaccggagaatatcgctggccaaagccttagcgtaggattccgccccttcccgcaaacgaccccaaacaggaaacgcagctgaaacgggaagctcaacacccactgacgcatgggttgttcaggcagtacttcatcaaccagcaaggcggcactttcggccatccgccgcgccccacagctcgggcagaaaccgcgacgcttacagctgaaagcgaccaggtgctcggcgtggcaagactcgcagcgaacccgtagaaagccatgctccagccgcccgcattggagaaattcttcaaattcccgttgcacatagcccggcaattcctttccctgctctgccataagcgcagcgaatgccgggtaatactcgtcaacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatagatctgcgggcagtgagcgcaacgcaattaatgtgagttagctcactcattaggcaccccaggctttacactttatgcttccggctcgtataatgtgtggaattgtgagcggataacaatttcacacaggatctagaaataattttgtttaactttaagaaggagatatacatatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaaccgtcgaagcctgtaaagcggcggtgcacaatcttctcgcgcaacgcgtcagtgggctgatagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaggatccggggggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagagcttgccgccgccgcaggtggagctgacttcatccgcacacacgagccgcgccccttgcgcgacgggctggcggtattggcggcgctgaaagaaaccgcaa

SEQ ID NO: 28 - protein, 264 aa, Escherichia coli
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 29 - DNA, 37 bp, Artificial sequence
  Description of artifical sequenceprimer
cctttggtaccagatctgcgggcagtgagcgcaacgc

SEQ ID NO: 30 - DNA, 36 bp, Artificial sequence
  Description of artificial sequenceprimer
aattgggatccgctcactgcccgctttccagtcggg

SEQ ID NO: 31 - DNA, 38 bp, Artificial sequence
  Description of artificial sequenceprimer
cgcttggatccggggggtggcccgatgaagaacgacag

SEQ ID NO: 32 - DNA, 33 bp, Artificial sequence
  Description of artificial sequenceprimer
ctcttggtaccgcctgatatacacgtcattgcc

SEQ ID NO: 33 - DNA, 33 bp, Artificial sequence
  Description of artificial sequenceprimer
tagcgagatctctgatgtccggcggtgcttttg

SEQ ID NO: 34 - DNA, 32 bp, Artificial sequence
  Description of artificial sequenceprimer
aaaaagagctcttacgccccgccctgccactc

SEQ ID NO: 35 - DNA, 31 bp, Artificial sequence
  Description of artificial sequenceprimer
cctttgagctcgcgggcagtgagcgcaacgc

SEQ ID NO: 36 - DNA, 34 bp, Artificial sequence
  Description of artificial sequenceprimer
ctgtttctagatcctgtgtgaaattgttatccgc

SEQ ID NO: 37 - DNA, 61 bp, Artificial sequence
  Description of artificial sequenceprimer
gcagggcctgtctcggtcgatcattcagcccggctcatagatctctgatgtccggcggtgc

SEQ ID NO: 38 - DNA, 26 bp, Artificial sequence
  Description of artificial sequenceprimer
cggaattcctgattggttacggcgcg

SEQ ID NO: 39 - DNA, 29 bp, Artificial sequence
  Description of artificial sequenceprimer
cccaagcttcgtaattagatagccaccgg

SEQ ID NO: 40 - DNA, 33 bp, Artificial sequence
  Description of artificial sequenceprimer
ggtgcctgggtgcatgatataatcatggggcaa

SEQ ID NO: 41 - DNA, 28 bp, Artificial sequence
  Description of artificial sequenceprimer
gccccatgataatatcatgcacccaggc

SEQ ID NO: 42 - DNA, 28 bp, Artificial sequence
  Description of artificial sequenceprimer
ctgtggtttctccagggcgacactaaca

SEQ ID NO: 43 - DNA, 28 bp, Artificial sequence
  Description of artificial sequenceprimer
tgttagtgtcgccctggagaaaccacag

SEQ ID NO: 44 - DNA, 795 bp, Escherichia coli
  CDS (1)..(795): thyA
atgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaa

SEQ ID NO: 45 - protein, 264 aa, Escherichia coli; identical to SEQ ID NO: 28

SEQ ID NO: 46 - DNA, 618 bp, Escherichia coli
  CDS (1)..(618)
atggcacagctatatttctactattccgcaatgaatgcgggtaagtctacagcattgttgcaatcttcatacaattaccaggaacgcggcatgcgcactgtcgtatatacggcagaaattgatgatcgctttggtgccgggaaagtcagttcgcgtataggtttgtcatcgcctgcaaaattatttaaccaaaattcatcattatttgatgagattcgtgcggaacatgaacagcaggcaattcattgcgtactggttgatgaatgccagtttttaaccagacaacaagtatatgaattatcggaggttgtcgatcaactcgatatacccgtactttgttatggtttacgtaccgattttcgaggtgaattatttattggcagccaatacttactggcatggtccgacaaactggttgaattaaaaaccatctgtttttgtggccgtaaagcaagcatggtgctgcgtcttgatcaagcaggcagaccttataacgaaggtgagcaggtggtaattggtggtaatgaacgatacgtttctgtatgccgtaaacactataaagaggcgttacaagtcgactcattaacggctattcaggaaaggcatcgccacgattaa

SEQ ID NO: 47 - protein, 205 aa, Escherichia coli
MAQLYFYYSAMNAGKSTALLQSSYNYQERGMRTVVYTAEIDDRFGAGKVSSRIGLSSPAKLFNQNSSLFDEIRAEHEQQAIHCVLVDECQFLTRQQVYELSEVVDQLDIPVLCYGLRTDFRGELFIGSQYLLAWSDKLVELKTICFCGRKASMVLRLDQAGRPYNEGEQVVIGGNERYVSVCRKHYKEALQVDSLTAIQERHRHD

SEQ ID NO: 48 - DNA, 7965 bp, Escherichia coli
aactgcacattcgggatatttctctatattcgcgcttcatcagaaaactgaaggaacctccattgaatcgaactaatattttttttggtgaatcgcattctgactggttgcctgtcagaggcggagaatctggtgattttgtttttcgacgtggtgacgggcatgccttcgcgaaaatcgcacctgcttcccgccgcggtgagctcgctggagagcgtgaccgcctcatttggctcaaaggtcgaggtgtggcttgccccgaggtcatcaactggcaggaggaacaggagggtgcatgcttggtgataacggcaattccgggagtaccggcggctgatctgtctggagcggatttgctcaaagcgtggccgtcaatggggcagcaacttggcgctgttcacagcctatcggttgatcaatgtccgtttgagcgcaggctgtcgcgaatgttcggacgcgccgttgatgtggtgtcccgcaatgccgtcaatcccgacttcttaccggacgaggacaagagtacgccgctgcacgatcttttggctcgtgtcgaacgagagctaccggtgcggctcgaccaagagcgcaccgatatggttgtttgccatggtgatccctgcatgccgaacttcatggtggaccctaaaactcttcaatgcacgggtctgatcgaccttgggcggctcggaacagcagatcgctatgccgatttggcactcatgattgctaacgccgaagagaactgggcagcgccagatgaagcagagcgcgccttcgctgtcctattcaatgtattggggatcgaagcccccgaccgcgaacgccttgccttctatctgcgattggaccctctgacttggggttgatgttcatgccgcctgtttttcctgctcattggcacgtttcgcaacctgttctcattgcggacaccttttccagcctcgtttggaaagtttcattgccagacgggactcctgcaatcgtcaagggattgaaacctatagaagacattgctgatgaactgcgcggggccgactatctggtatggcgcaatgggaggggagcagtccggttgctcggtcgtgagaacaatctgatgttgctcgaatatgccggggagcgaatgctctctcacatcgttgccgagcacggcgactaccaggcgaccgaaattgcagcggaactaatggcgaagctgtatgccgcatctgaggaacccctgccttctgcccttctcccgatccgggatcgctttgcagctttgtttcagcgggcgcgcgatgatcaaaacgcaggttgtcaaactgactacgtccacgcggcgattatagccgatcaaatgatgagcaatgcctcggaactgcgtgggctacatggcgatctgcatcatgaaaacatcatgttctccagtcgcggctggctggtgatagatcccgtcggtctggtcggtgaagtgggctttggcgccgccaatatgttctacgatccggctgacagagacgacctttgtctcgatcctagacgcattgcacagatggcggacgcattctctcgtgcgctggacgtcgatccgcgtcgcctgctcgaccaggcgtacgcttatgggtgcctttccgcagcttggaacgcggatggagaagaggagcaacgcgatctagctatcgcggccgcgatcaagcaggtgcgacagacgtcatactagatatcaagcgacttctcctatcccctgggaacacatcaatctcaccggagaatatcgctggccaaagccttagcgtaggattccgccccttcccgcaaacgaccccaaacaggaaacgcagctgaaacgggaagctcaacacccactgacgcatgggttgttcaggcagtacttcatcaaccagcaaggcggcactttcggccatccgccgcgccccacagctcgggcagaaaccgcgacgcttacagctgaaagcgaccaggtgctcggcgtggcaagactcgcagcgaacccgtagaaagccatgctccagccgcccgcattggagaaattcttcaaattcccgttgcacatagcccggcaattcctttccctgctctgccataagcgcagcgaatgccgggtaatactcgtcaacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatagatctgcgggcagtgagcgcaacgcaattaatgtgagttagctcactcattaggcaccccaggctttacactttatgcttccggctcgtataatgtgtggaattgtgagcggataacaatttcacacaggatctagaaataattttgtttaactttaagaaggagatatacatatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaaccgtcgaagcctgtaaagcggcggtgcacaatcttctcgcgcaacgcgtcagtgggctgatagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagctaatggatcaccgcaaacaggttactcgcctggggattccctttcgacccgagcatccgtatgatactcatgctcgattattattattatagaagcccccatgaataaatcgctcatcattttcggcatcgtcaacataacctcggacagtttctccgatggaggccggtatctggcgccagacgcagccattgcgcaggcgcgtaagctgatggccgagggggcagatgtgatcgacctggtccggcatccagcaatcccgacgccgcgcctgtttcgtccgacacagaaatcgcgcgtatgcgccggtgctggacgcgctcaggcagatggcattcccgtctcgctcgacagttatcaacccgcgacgcaagcctatgccttgtcgcgtggtgtggcctatctcaatgatattcgcggttttccagacgctgcgttctatccgcaattggcgaaatcatctgccaaactcgtcgttatgcattcggtgcaagacgggcaggcagatcggcgcgaggcacccgctggcgacatcatggatcacattgcggcgttctttgacgcgcgcatcgcggcgctgacgggtgccggtatcaaacgcaaccgccttgtccttgatcccggcatggggttttttctgggggctgctcccgaaacctcgctctcggtgctggcgcggttcgatgaattgcggctgcgcttcgatttgccggtgcttctgtctgtttcgcgcaaatcctttctgcgcgcgctcacaggccgtggtccgggggtgtcggggccgcgacactcgctgcagagcttgccgccgccgcaggtggagctgacttcatccgcacacacgagccgcgccccttgcgcgacgggctggcggtattggcggcgctgaaagaaaccgcaagaattcgtt

(19) United States
(12) Patent Application Publication
(10) Pub. No.: US 2006/0014257 A1
(43) Pub. Date: Jan. 19, 2006

(54) RSF1010 derivative Mob' plasmid containing no antibiotic resistance gene, bacterium comprising the plasmid and method for producing useful metabolites

(76) Inventors: Joanna Yosifovna Katashkina, Moscow (RU); Aleksandra Yurievna Skorokhodova, Moscow (RU); Danila Vadimovich Zimenkov, Moscow (RU); Andrey Yurievich Gulevich, Moscow (RU); Lopes Lubov Errais, Moscow (RU); Irina Vladimirovna Biryukova, Moscow (RU); Aleksandr Sergeevich Mironov, Moscow (RU); Sergei Vladimirovich Mashko, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/165,067

(22) Filed: Jun. 24, 2005

(30) Foreign Application Priority Data
Jun. 24, 2004 (RU) 2004119027

(51) Int. Cl. C12P 19/28; C12P 13/04; C12N 15/74; C12N 1/21

(52) U.S. Cl. 435/85; 435/106; 435/252.3; 435/471

(57) ABSTRACT

A Mob⁻ plasmid having a RSF1010 replicon, comprising a gene coding for Rep protein and said plasmid has been modified to inactivate gene related to mobilization ability. The present invention also describes a bacterium having an ability to produce useful metabolites, comprising the plasmid and said bacterium lack active thymidylate synthase coded by thyA gene and thymidine kinase coded by tdk gene, and a method for producing useful metabolites, such as native or recombinant proteins, enzymes, L-amino acids, nucleosides and nucleotides, vitamins, using the bacterium.

### BACKGROUND OF THE INVENTION

1. Field of the Invention

The present invention relates to a mutant vector and its uses, and more specifically, a broad host range RSF1010 derivative Mob⁻ plasmid containing no antibiotic resistance gene. The present invention also relates to a bacterium comprising the plasmid and a method of using the bacterium for producing useful metabolites.

2. Brief Description of the Related Art

RSF1010 is a mobilizable, but not self-transmissible, well-known plasmid of the IncQ group which has a remarkable capability to replicate in a broad range of bacterial hosts, including most of the gram-negative bacteria (Frey, J. and Bagdasarian, M. The molecular biology of IncQ plasmids. In: Thomas, C. M. (Ed.), Promiscuous Plasmids of Gram Negative Bacteria. Academic Press, London, 1989, p. 79-94). The nucleotide sequence of the RSF1010 plasmid is known (Scholz, P. et al, Gene, 75 (2), 271-288 (1989); accession number in GenBank M28829, gi:152577) and the functional structure of the plasmid has been fairly thoroughly investigated. The RSF1010 plasmid contains oriV, the unique origin of vegetative DNA replication (De Graaf, J. et al, J. Bacteriol., 134, 1117-1122 (1978); Haring, V. and Scherzinger, E, Replication Proteins of the IncQ plasmid RSF1010, In:Thomas, C. M. (Ed.), Promiscuous Plasmids of Gram Negative Bacteria. Academic Press, London, 1989, p. 95-124), as well as repA, repB, repB′ and repC, which are the genes essential for the replication of the plasmid (Scherzinger, E et al, Proc. Natl. Acad. Sci. USA, 81, 654-658 (1984); Scherzinger, E et al, Nucleic Acids Res., 19, 1203-1211 (1991); Scholz, P. et al, Replication determinants of the broad-host-range plasmid RSF1010. In: Helinski, D. R. et al (Eds), Plasmids in Bacteria, Plenum Press, New York, 1984, p. 243-259). The RSF1010 plasmid also contains oriT, the site of the relaxation complex and the origin of conjugative DNA transfer, mobA (including repb gene in the alternative frame), mobB and mobC (mob locus), genes encoding trans-active proteins, which are involved in the plasmid mobilization (Nordheim, A et al, J. Bacteriol., 144, 923-932 (1980); Derbyshire. K. M. et al, Mol. Gen. Genet., 206, 161-168 (1987)), as well as the sulfonamide resistance (Sul^{R}) and streptomycin resistance (Str^{R}) genes (sul and str genes, respectively) (Scholz, P. et al, Gene, 75 (2), 271-288 (1989)).

Promoters which cause translation of the plasmid proteins on the RSF1010 physical map were recognized by electron microscopy (Bagdasarian, J. Frey, and K. Timmis. Gene 16, 237-247 (1981)) and confirmed once the plasmid sequence was completed (Scholz, P. et al, Gene, 75 (2), 271-288 (1989)).

The initiation of replication of the RSF1010 plasmid requires the presence of three proteins encoded by the plasmid: RepA, RepB and RepC, encoded by the repA, repB and repC genes, respectively. RepC recognizes the origin of replication (in the repeat sequences) and positively regulates initiation of replication; RepA has helicase activity; RepB and RepB* (which correspond to two proteins encoded by the same frame but are each initiated at a different codon) have RSF1010-specific primase activity in vitro. The replication of the RSF1010 plasmid is dependent on DNA polymerase III and the gyrase of the host. The RSF1010 plasmid may be mobilized from one Gram-negative bacterium to another Gram-negative bacterium by the tra functions of the plasmids of the incompatibility groups IncI-α, IncM, IncX and most especially IncP (Derbyshire. K. M. et al, Mol. Gen. Genet., 206, 161-168 (1987)).

In E. coli, RSF1010 is present at a copy number of 12 per cell (Bagdasarian, M. M. et al, Regulation of the rep operon expression of the broad-host-range plasmid RSF1010. In: Novick, R and Levy, S (Eds.), Evolution and Environmental Spread of Antibiotic Resistance Genes. Cold Spring Harbor Laboratory, Cold Spring Harbor, N.Y., 1986, p. 209-223). The structural organization of the oriT region of the plasmid, which is placed between mobC and mobB genes, is rather complicated. However, it is known that this region is necessary for mobilization initiation and also contains promoters essential for plasmid replication. It has been shown that the elimination of separate genes involved in plasmid mobilization may unpredictably change the plasmid properties. For example, deletion of the mobC gene, which encodes the regulatory protein, leads to a significant increase in the copy number of the plasmid (Frey, J. et al, Gene, 113, 101-106 (1992)). This could be the reason why variants of RFS1010 plasmid, which do not contain all of the known sequences essential for mobilization, are still not known.

No study relating to the stability of RSF1010 and its derivatives has been described to date. Furthermore, although the sequence of RSF1010 is known, no determinant of plasmid stability has been able to be identified, either by functional analysis or by molecular analysis.

The constraints of biosafety oblige recombinant strains to be greatly confined biologically. The biosafety level 1 (BLI) system described in “Guidelines for research involving recombinant DNA molecules” published by the NIH on the 7th of May, 1987 corresponds to some of these constraints. If, for example, the recombinant microorganism were to be accidentally released into the natural environment, it is imperative that such plasmids cannot be transmitted to other organisms. A similar regulation is stated in the European directives; such as Council Directive of 23 Apr. 1990 on the deliberate release into the environment of genetically modified organisms (90/220/EEC), Council Directive 98/81/EC of 26 Oct. 1998 amending Directive 90/219/EEC on the contained use of genetically modified microorganisms.

A Gram-negative bacterial vector comprising an origin of replication which is functional in Gram negative bacteria, the par region of the plasmid RP4, and lacking the mobilization functions have been disclosed (U.S. Pat. No. 5,670,343). The vectors of the present invention are not mobilizable from one Gram-negative bacterium to another. Hence, they form class 1 host-vector systems with these bacteria and comply with industrial regulations. This system, both in Escherichia coli and in Pseudomonas putida, assumes the use of non-conjugative and non-mobilizable plasmids. This very advantageous property of the vectors of the present invention was obtained, in particular, by deleting a region containing the mob locus. Such new cloning and/or expression vectors having a broad host range in Gram-negative bacteria could be used in the production of recombinant proteins or metabolites by host cells containing such vectors.

To date the genetic engineering of microorganisms has depended almost entirely on the use of antibiotic resistance genes, either to genetically label recipient cells or to identify and maintain plasmids used as vectors in genetic engineering protocols. The release of genetically modified organisms (GMO) into the general environment, their use in agriculture and food processing industries or their use in health care industries is likely to be curtailed by regulatory agencies if the strains carry antibiotic resistance genes. There is an obvious need, therefore, for marker genes which can be used in place of antibiotic resistance genes and which will not have any consequence which might slow clearance by regulatory agencies of GMO carrying the substitute marker genes.

Previously, the thymidylate synthase (TS) gene was described as being suitable to replace antibiotic resistance genes as a selection marker (European patent application EP0406003A1). In particular, the thymidylate synthase gene from Streptococcus lactis, a species of bacteria routinely used for cheese manufacture (and therefore established as a safe microbe) was found to be a suitable candidate as a marker gene which can be a substitute for antibiotic resistance genes, especially as a “food grade” marker gene. Thymidylate synthase (5,10-methylenetetrahydrofolate:dUMP C-methyl-transferase; EC 2.1.1.45) plays a key role in DNA synthesis; it catalyses the reductive methylation of dUMP to dTMP with concomitant conversion of the cofactor 5,10-methylenetetrahydrofolic acid to 7,8-dihydrofolic acid. This activity is an essential step in de novo biosynthesis of DNA. Cells which have lost TS activity, through mutation in the TS gene, cannot make DNA and cannot survive unless supplied with thymine or thymidine, which is converted to dTMP by an alternative pathway. Strains of microorganisms devoid of thymidylate synthase activity (i.e. TS⁻) can easily be distinguished from normal TS⁺ strains. In chemically defined growth media, which support positive growth of TS⁺ strains, TS⁻ cells die unless the medium is supplemented with thymine or thymidine. Furthermore, cloned vector plasmids with the S. lactis TS gene will be stably maintained in TS⁻ cells in media or environments which do not have sufficient thymine or thymidine, as loss of the plasmid results in cell death.

### SUMMARY OF THE INVENTION

An object of the present invention is to provide a broad host range Mob⁻ vector derived from RSF1010 plasmid containing no antibiotic resistance gene, to provide bacterium comprising the vector and lacking activity of thymidylate synthase and thymidine kinase providing the very stabile vector-host system, and to provide a method for producing useful metabolites using the bacterium.

This aim was achieved by constructing a RSF1010 derivative plasmid containing no genes related to mobilization ability and having no antibiotic resistance genes. Further, the thymidylate synthase gene as a selection marker was introduced into the constructed plasmid. And further, the bacterium lacking active thymidylate synthase and thymidine kinase genes was transformed with said plasmid. As a result the thymidylate synthase gene existing on the plasmid became not only selection marker but also the factor for stabilization of the plasmid in the bacterium. Thus the present invention has been completed.

It is an object of present invention to provide a RSF1010 derivative Mob- plasmid, wherein said plasmid is selected from the group coiisising of SEQ ID NO: 24, SEQ ID NO: 27, and SEQ ID NO: 48 and variants of SEQ ID NO: 24, SEQ ID NO: 27 and SEQ ID NO: 48 which are at least 95% homologous to SEQ ID NO: 24, SEQ ID NO: 27, and SEQ ID NO: 48, and wherein said plasmid has been modified to inactivate a gene or genes related to mobilization ability.

It is a further object of the present invention to provide the plasmid described above, wherein the plasmid has been modified to inactivate an antibiotic resistance gene.

It is a further object of the present invention to provide the plasmid described above, wherein the plasmid has been modified to increase the copy number of the plasmid.

It is a further object of the present invention to provide the plasmid described above, comprising a PlacUV5 promoter and an origin of replication from RSF1010 without a mob locus.

It is a further object of the present invention to provide the plasmid described above, additionally comprising a thymidylate synthase gene.

It is a further object of the present invention to provide the plasmid described above, additionally comprising a gene of interest.

It is a further object of the present invention to provide the bacterium comprising the plasmid described above.

It is a further object of the present invention to provide the bacterium described above, wherein said bacterium is a Gram negative bacterium.

It is a further object of the present invention to provide the bacterium described above, wherein said bacterium lacks active thymidylate synthase and lacks active thymidine kinase.

It is a further object of the present invention to provide the bacterium described above, wherein said bacterium has an ability to produce a useful metabolite.

It is a further object of the present invention to provide the bacterium described above, wherein said useful metabolite is selected from the group consisting of native or recombinant proteins, enzymes, L-amino acids, nucleosides, nucleotides, organic acids and vitamins.

It is a further object of the present invention to provide a method for producing a useful metabolite, comprising
(a) cultivating the bacterium described above in a culture medium and
(b) collecting said useful metabolite from the culture medium.

It is a further object of the present invention to provide a method described above, wherein said useful metabolite is selected from the group consisting of native or recombinant proteins, enzymes, L-amino acids, nucleosides, nucleotides, organic acid and vitamins.

### BRIEF DESCRIPTION OF THE DRAWINGS

FIG. 1 shows the structure of RSF1010 plasmid.

FIG. 2 shows the structure of pBluescript::lacIrepB plasmid.

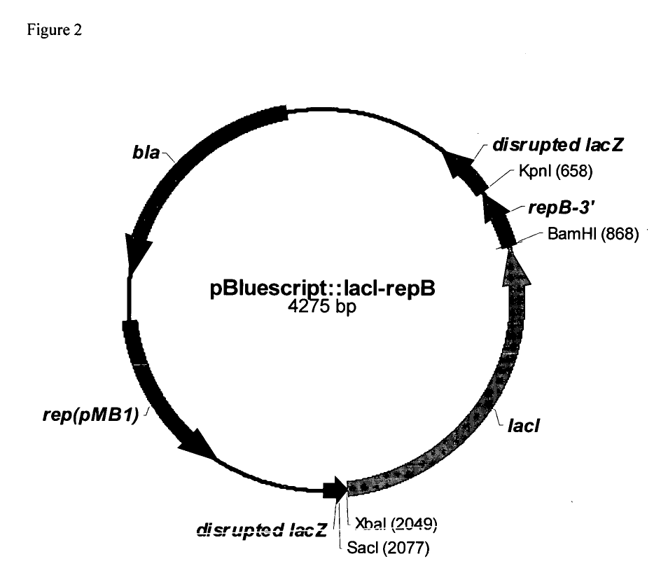

FIG. 3 shows the structure of RSF1010mob⁻ plasmid.

FIG. 4 shows sequence of wild type and improved thyA promoter region. −35 and −10 regions are underlined. Substitutions in −10, −14 and −15 regions are in bold.

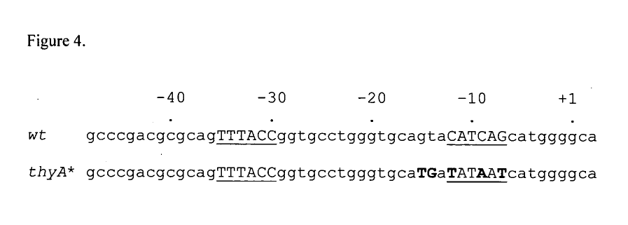

FIG. 5 shows the structure of RSF1010-MT plasmid.

### DESCRIPTION OF THE PREFERRED EMBODIMENTS

The RSF1010 derivative Mob⁻ plasmid of the present invention encompasses a plasmid constructed from the RSF1010 plasmid whereby the genes related to mobilization ability were inactivated.

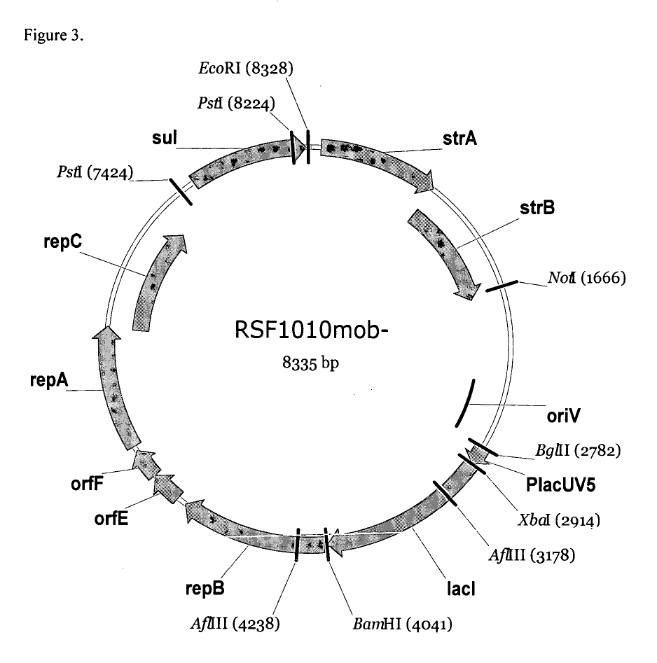

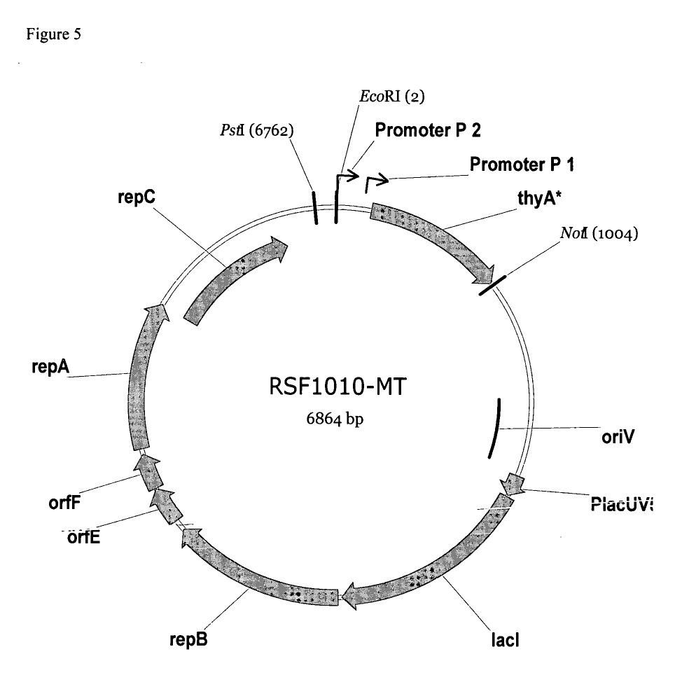

The phrase “RSF1010 derivative Mob⁻ plasmid” as used in the present invention is defined as the RSF1010 plasmid as defined below and in SEQ ID NO. 1, and variants thereof, whereby the genes related to mobilization ability were inactivated. The examples of RSF1010 derivative Mob⁻ plasmid is presented in FIG. 3, FIG. 5, and the DNA sequences are disclosed in SEQ ID NO:24, 27 and 48.

The phrase “derivative” of a plasmid means another plasmid composed of a part of the plasmid and/or another DNA sequenc. “A part of the plasmid” means a part containing a region essential for autonomous replication of the plasmid such as replication origin (ori) and a gene necessary for replication (rep) in order to maintain replication in a bacteria.

The genes related to mobilization include, but are not limited to mobA, mobB, mobC, and oriT. Location of genes included in the plasmid RSF1010 are shown in Table 1.

TABLE 1

| Gene | Protein | Sequence (SEQ ID:1) | SEQ ID NO: |
|---|---|---|---|
| strA | Sm resistance protein A | 63-866 | NO2 |
| strB | Sm resistance protein B | 866-1702 | NO4 |
| oriV | origin of repliation | 2347-2771 | |
| mobC | mobilization protein C | complement | NO6 |
| | | 2767-3051 | |
| mobA | mobilization protein A | 3250-5379 | NO8 |
| mobB | mobilization protein B | 3998-4411 | NO10 |
| repB | replication protein B | 4408-5379 | NO12 |
| orfE | unknown protein E | 5440-5652 | NO14 |
| orfF | repressor protein F | 5654-5860 | NO16 |
| repA | replication protein A | 5890-6729 | NO18 |
| repC | replication protein C | 6716-7567 | NO20 |
| suI | Su resistance protein | 7875-8663 | NO22 |

The nucleotide sequence of the RSF1010 plasmid is known (Scholz, P. et al, Gene, 75 (2), 271-288 (1989); accession number in GenBank M28829, gi:152577) and depicted in SEQ ID NO: 1. The RSF1010 plasmid contains oriV, the unique origin of vegetative DNA replication, repA, repB, repB′ and repC, the genes which encode the essential replication proteins, oriT, the relaxation complex site and conjugative DNA transfer origin, mobA, mobB and mobC, genes which encode the trans-active proteins involved in plasmid mobilization, as well as the sulfonamide and streptomycin resistance (Str^{R}) genes (sul and stra, strB genes, respectively).

The RSF1010 plasmid comprises genes coding for Rep protein having amino acid sequences shown in SEQ ID:13, 19 and 21.

The Rep genes are repA, B, C genes from RSF1010 or a homologue thereof. RepA, B, C genes include genes encoding a protein having an amino acid sequence SEQ ID NOS: 13, 19, 21. The rep gene homologue may be a gene encoding a protein having a homology of 70% or more, preferably 80% or more, more preferably 90% or more, more preferably 95% or more, particularly preferably 98% or more, to the total amino acid sequence of SEQ ID NO: 13, 19, and 21, and having replication ability. The homology of amino acid sequence and DNA sequence can be determined using the algorithm BLAST (Pro. Natl. Acad. Sci. USA, 90, and 5873 (1993)) and FASTA (Methods Enzymol., 183, and 63 (1990)) by Karlin and Altschul. The program called BLASTN and BLASTX is developed based on this algorithm BLAST. (refer to http://www.ncbi.nlm.nih.gov).

Furthermore, the rep gene of the present invention is not limited to a wild-type gene, but may be a mutant or artificially modified gene encoding a protein having an amino acid sequence of SEQ ID NO: 13, 19 and 21. The encoded protein may include substitutions, deletions, or insertions, of one or several amino acid residues at one or more positions so long as the function of the encoded Rep protein, namely, replication ability, is maintained. Although the number of “several” amino acid residues referred to herein differs depending on positions in the three-dimensional structure or types of amino acid residues, it may be 2 to 20, preferably 2 to 10, more preferably 2 to 5. Substitution of amino acids is preferably a conserved substitution including substitution of ser or thr for ala, substitution of gin, his or lys for arg, substitution of glu, gin, lys, his or asp for asn, substitution of asn, glu or gin for asp, substitution of ser or ala for cys, substitution of asn, glu, lys, his, asp or arg for gin, substitution of gly, asn, gin, lys or asp for glu, substitution of pro for gly, substitution of asn, lys, gin, arg or tyr for his, substitution of leu, met, val or phe for ile, substitution of ile, met, val or phe for leu, substitution of asn, glu, gin, his or arg for lys, substitution of ile, leu, val or phe for met, substitution of trp, tyr, met, ile or leu for phe, substitution of thr or ala for ser, substitution of ser or ala for thr, substitution of phe or tyr for trp, substitution of his, phe or trp for tyr and substitution of met, ile or leu for val. The substitution, deletion, or insertion, of one or several nucleotides as described above also includes a naturally occurring mutation arising from individual differences, and differences in species of microorganisms that harbor the rep gene (mutant or variant).

Such genes can be obtained by modifying a nucleotide sequence shown in SEQ ID NOS: 12, 18 and 20 by, for example, site-specific mutagenesis, so that one or more substitutions, deletions, or insertions are introduced at a specific site of the protein encoded by the gene.

Furthermore, such genes can also be obtained by conventional mutagenesis treatments such as those mentioned below. Examples of mutagenesis treatments include treating a gene having a nucleotide sequence shown in SEQ ID NOS: 12, 18 and 20 in vitro with hydroxylamine, and treating a microorganism such as an Escherichia bacterium harboring the RSF1010 with ultraviolet ray irradiation or a mutagenesis agent used in a typical mutation treatments such as N-methyl-N′-nitro-N-nitrosoguanidine (NTG) or EMS (ethyl methanesulfonate).

The rep gene also includes a DNA which is able to hybridize under stringent conditions with a nucleotide sequence of SEQ ID NOS: 12, 18 and 20, or a probe prepared from these sequences, and which encodes a protein having replication ability. “Stringent conditions” as used herein are conditions under which a so-called specific hybrid is formed, and a non-specific hybrid is not formed. It is difficult to clearly express this condition by using any numerical value. However, examples of stringent conditions include, those under which DNAs having high homology to each other, for example, DNAs having a homology of not less than 50%, hybridize to each other, and DNAs having homology lower than 50% do not hybridize to each other, and those under which DNAs hybridize to each other at a salt concentration with washing typical of Southern hybridization, i.e., washing once or preferably 2-3 times under 1×SSC, 0.1% SDS at 60° C., preferably 0.1×SSC, 0.1% SDS at 60° C., more preferably 0.1×SSC, 0.1% SDS at 68° C.

A DNA coding for a mobilization protein or other genes used in this invention can be obtained following similar procedures for Rep protein, as described above.

The phrase “inactivate a gene or genes related to mobilization ability” as used herein means to lose mobilization activity from cell to another cell. The gene related to mobilization ability includes mobA and mobB and mobC. Examples of methods of inactivating gene include mutating or deleting a part of gene selected from mobA, B, and C. Examples of methods of mutating or deleting a gene include modification of expression regulatory sequences such as promoters and Shine-Dalgarno (SD) sequences, introduction of mis-sense mutations, non-sense mutations, or frame-shift mutations into an open reading frame, and deletion of a portion of the gene (J. Biol. Chem. 1997 272(13):8611-7), or deletion of the entire region which encodes for a mobilization protein. A mutated gene can be introduced into a microorganism by using a homologous recombination technique in which a wild-type gene on a chromosome is replaced with the mutated gene, or by using a transposon or IS factor. Homologous recombination techniques include methods using linear DNA, a temperature-sensitive plasmid, and non-replicable plasmid. These methods are described in Proc. Natl. Acad. Sci. USA. 2000 Jun. 6; 97(12):6640-5, U.S. Pat. No. 6,303,383, JP05-007491A, and the like.

The mobA gene contains the mobB gene in the alternative frame and the 3′-end of the mobA gene encodes for the RepB protein, which is essential for plasmid replication. Moreover, the start codon of the repB gene overlaps with the stop codon of the mobB gene, assuming that the translation coupling of these genes exists.

The oriT region of the plasmid exists between the mobC and the mobB genes, and is an element necessary for mobilization initiation. It is known that this region also contains promoters essential for repB gene translation. It is necessary, therefore, to introduce another promoter(s) which can function for repB gene translation.

Deletion of parts of the plasmid can be performed by conventional methods for constructing recombinant plasmids, such as digestion with restriction enzymes followed by ligation of a remaining part of the plasmid, recombination, or integration, and so on.

The particular embodiment of the present invention is the RSF1010 derivative plasmid which has the mobA, mobB and mobC genes deleted. The mobA gene extends from nucleotide 3250 to nucleotide 5379, the mobB gene extends from nucleotide 3998 to nucleotide 4411, the mobC gene extends from nucleotide 3051 to nucleotide 2767 on the original RSF1010 plasmid (SEQ ID NO: 1). The coding region of repB and mobA is overlapped. So it is preferable to delete mobA without deleting repB such as nucleotides 3250-5379. The sequence of the RSF1010 derivative minus the mob locus, RSF1010 derivative Mob⁻, is presented in the Sequence Listing in SEQ ID NOS: 24, 27 and 48.

A further embodiment of the present invention is the RSF1100 derivative Mob⁻ plasmid which comprises no antibiotic resistance marker. The original RSF1010 plasmid contains streptomycin resistance genes (strA and strB genes) and sulfonamide resistant gene (sul gene). The strA gene extends from nucleotide 63 to 866, the strB gene extends from nucleotide 866 to 1702, the sul gene extends from nucleotide 7875 to 8663 on the RSF1010 plasmid (SEQ ID NO: 1). The RSF1010 derivative Mob⁻ plasmid which comprises no antibiotic resistance marker is presented in SEQ ID NO: 27 and FIG. 5.

A further embodiment of the present invention is the RSF1010 derivative Mob⁻ plasmid wherein the plasmid has been modified to inactivate an antibiotic resistance gene. The gene related to an antibiotic resistance gene in this invention includes sulfonamide and streptomycin resistance (StrR) genes (sul and stra, strB genes, respectively).

A further embodiment of the present invention is the RSP1010 derivative Mob- plasmid, wherein the plasmid has been modified to increase the copy number of the plasmid. A strong promoter or inducible promoter can be used for expression of the repB gene, which is modified so that the copy number of the plasmid can be increased. Examples of such strong promoters include lac promoter, trp promoter, trc promoter, tac promoter, PR promoter and PL promoter of lambda phage, tet promoter, amyE promoter, spac promoter, and so forth. Examples of such strong promoters include P_{lacUV5} promoter, lac promoter, especially P_{lacUV5} promoter is preferable. The RSF1010 derivative mob- plasmid comprising P_{lacUV5} promoter is described in SEQ ID NOS: 24, 27 and 48.

In order to conditionally regulate the copy number, the combination P_{lacUV5} promoter and the lacI gene under control of the P_{lacUV5} promoter can be used. P_{lacUV5} promoter is inducible by IPTG addition and the expression from P_{lacUV5} promoter is repressed by the lad gene (J Mol. Biol. 1982 Nov. 5; 161(3):417-38.); therefore, in order to increase copy number, IPTG can be added, or lacI gene can be deleted. It is desirable for copy numbers of this plasmid to increase up to twice, 3 times, and 4 times compared with RSF1010. In order to decrease the copy number of plasmid, it is preferable that lacI gene is modified to be overexpressed. The RSF1010 mob- lacd- plasmid comprising the P_{lacUV5} promoter is described in SEQ ID NO: 48.

In order to decrease the copy number of plasmid, it is preferable that lacI gene is modified to be overexpressed. The nucleotide sequence of P_{lacUV5} promoter is disclosed in Genbank Accession No. Y00412 (nucleotides 7-100). The nucleotide sequence of lacI is disclosed in Genbank Accession No. NP_{—}414879. Furthermore, the nucleotide sequence Of P_{lacUV5} promoter used in the present invention is described in SEQ ID NO: 24 (nucleotides 2824-2912). The P_{lacUV5} promoter can be obtained by chemical synthesis according to the nucleotide sequence of SEQ ID NO: 24, or by preparing from the pET Expression System (Novagen). The nucleotide sequence of lacI is also described in SEQ ID NO: 25. The lacI can be obtained by PCR according to the nucleotide sequence of SEQ ID NO: 25 or GenBank Accession No. NP 414879 using chromosomal DNA of E. coli K-12 (MG1655) as a template. The RSF1010mob- plasmid comprising P_{lacUV5} promoter and lacI is presented in SEQ ID NO: 24 and FIG. 3.

A further embodiment of the present invention is the RSF1010 derivative Mob⁻ plasmid additionally containing thymidylate synthase gene (thyA gene, SEQ ID NO:44) as a selection marker. Thymidylate synthase catalyzes formation of thymidine-5′-monophosphate (dTMP) from 2′-deoxyuridine-5′-phosphate (dUMP) by consuming 5,10-methylenetetrahydrofolate upon the release of 7,8-dihydrofolate. The thyA gene, which encodes thymidylate synthase of Escherichia coli, has been elucidated (nucleotide numbers 2962383 to 2963177 in the sequence of GenBank accession NC_{—}000913.1, gi:16130731). The thyA gene is located between the ppdA and Igt genes on the chromosome of E. coli strain K12. Therefore, the aforementioned gene can be obtained by PCR (polymerase chain reaction; refer to White, T. J. et al., Trends Genet., 5, 185 (1989)) utilizing primers based on the reported nucleotide sequence of the gene. The sequence of the derivative of RSF1010 having mob locus and all antibiotic resistance genes deleted and containing thymidylate synthase gene (thyA gene, SEQ ID NO: 27 196 to 990) as a selection marker is presented in the Sequence Listing in SEQ ID NOS: 44 and 45.

The RSF1010 derivative Mob⁻ plasmid additionally containing thymidylate synthase gene (thyA gene) as a selection marker can be used as vector. Vector is a DNA molecule into which another DNA fragment of appropriate size can be integrated without loss of the vectors capacity for self-replication; vectors introduce foreign DNA into host cells, where it can be reproduced in large quantities. The plasmid containing thyA gene is described in SEQ ID NO: 27 (RSF1010mob-MT) and FIG. 5.

So, a further embodiment of the present invention is RSF1010 derivative Mob⁻ plasmid containing thymidylate synthase gene (thyA gene) as a selection marker and additionally containing a gene of interest. The term “gene of interest” means a gene which is involved in or influences the biosynthetic pathways of a useful metabolite. These could be genes involved in the biosynthesis of L-amino acids, nucleosides, nucleotides, organic acid and vitamins or genes coding for regulatory protein. The term “useful metabolite” includes native or recombinant proteins, enzymes, L-amino acids, nucleosides and nucleotides, organic acid, vitamins. L-amino acids include L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, L-valine and L-homoserine, and preferably includes aromatic L-amino acids, such as L-tryptophan, L-phenylalanine and L-tyrosine. Nucleosides include purine nucleosides and pyrimidine nucleosides, such as adenosine, cytidine, inosine, guanosine, thymidine, uridine and xanthosine. Nucleotides include phosphorylated nucleosides, preferably 5′-phosphorylated nucleosides, such as 2′-deoxyadenosine-5′-monophosphate (dAMP), 2′-deoxycytidine-5′-monophosphate (dCMP), 2′-deoxyguanosine 5′-monophosphate (dGMP), thymidine-5′-monophosphate (dTMP), adenosine-5′-monophosphate (AMP), cytidine-5′-monophosphate (CMP), guanosine 5′-monophosphate (GMP), inosine 5′-monophosphate (IMP), uridine-5′-phosphate (UMP), xanthosine-5′-monophosphate (XMP). Organic acids include succinate, fumarate, malate, ketogluconic acid. Vitamins include pantothenic acid.

The plasmids of the present invention, particularly, the plasmids shown in SEQ ID Nos. 24, 27 and 48, may include variants of these sequences, so long as the plasmid can function in the bacterium as compared to the plasmid prior to generation of the variants. The function of the plasmid as used herein means that the plasmid when transformed into a bacterium has the ability to replicate itself and express a gene of interest, as well as express the genes necessary for replication of the plasmid. The significant variations or even deletions can occur in regions of the plasmid which are not critical for the function and replication of the plasmid, such as regions from nucleotides 7219 to 8335 and nucleotides from 1 to 2347 for RSF1010 derivative mob- plasmid (SEQ ID NO: 24), and regions from nucleotides 1004 to 1649 and/or from 6557 to 6864 for RSF1010-MT plasmid (SEQ ID NO: 27). These regions usually may contain one or several markers for selection. Further, coding part of lacI gene necessary for regulation of the plasmid replication (nucleotides 2252 to 3379 for RSFmob⁻ plasmid (SEQ ID NO:24) and nucleotides 2914 to 4041 for RSF1010-MT plasmid (SEQ ID NO:27)) could be also modified or deleted (see Example 2) provided that such modification or deletion do not create stop-codons within lacI gene or do not generate frame shift. The further variations can be substitutions, deletions, or insertions of nucleotides in other regions of SEQ ID Nos. 24, 27 and 48, as long as the plasmid can function and replicate as it did prior to the generation of the variant. Preferably, the variants are at least 80% homologous when compared to the sequence of SEQ ID Nos. 24, 27 and 48, more preferably, at least 90% homologous, and most preferably, at least 95% homologous, and even most preferably, at least 97% homologous. Homology can be measured by ordinary and well-known techniques, such as BLAST, and is measured over the entire length of the sequence of SEQ ID NOs. 24, 27 and 48. For example, homology between two amino acid sequences could be estimated using software program BLAST 2.0 calculating three parameters: score, identity and similarity. And value of similarity obtained during calculation is taking in account to estimate the percentage of homology. BLAST (Basic Local Alignment Search Tool) is the heuristic search algorithm employed by the programs blasta, blastp, blastn, blastx, megablast, tblastn, and tblastx; these programs assign significance to their findings using the statistical methods of Karlin, Samuel and Stephen F. Altschul (“Methods for assessing the statistical significance of molecular sequence features by using general scoring schemes”. Proc. Natl. Acad. Sci. USA, 87:2264-68 (1990); “Applications and statistics for multiple high-scoring segments in molecular sequences”. Proc. Natl. Acad. Sci. USA, 90:5873-7 (1993)).

Methods for preparation of chromosomal DNA, hybridization, PCR, preparation of plasmid DNA, digestion and ligation of DNA, transformation, selection of an oligonucleotide as a primer and the like include ordinary methods well-known to those skilled in the art. These methods are described in Sambrook, J., and Russell D., “Molecular Cloning A Laboratory Manual, Third Edition”, Cold Spring Harbor Laboratory Press (2001) and the like.

The bacterium of present invention includes a bacterium containing the plasmid of the present invention, preferably a Gram-negative bacterium. Preferably, the bacterium of the present invention has an ability to produce a useful metabolite. Furthermore, the bacterium of the present invention includes a bacterium as described above, which lacks active inherent thymidylate synthase and thymidine kinase. However, the bacterium of the present invention may have active thymidylate synthase which is expressed from the plasmid of the present invention harbored by the bacterium.

The term “bacterium having an ability to produce a useful metabolite” means a bacterium, which has an ability to cause accumulation of the metabolite in a cell of the bacterium or, preferably, in a medium when the bacterium of the present invention is cultured in the medium. The ability to produce such metabolite may be imparted or enhanced by breeding. The term “bacterium having an ability to produce a useful metabolite” as used herein also means a bacterium, which is able to produce and cause accumulation of the metabolite in a culture medium in an amount larger than a wild-type or parental strain, and preferably means that the microorganism is able to produce and cause accumulation in a medium of the target metabolite in an amount not less than 0.5 g/L, more preferably not less than 1.0 g/L.

The term “Gram negative bacterium” means that the bacterium is classified as the Gram negative bacterium according to the classification known to a person skilled in the microbiology. For such classification see, for example, “Bergey's Manual of Determinative Bacteriology, Ninth edition” (by Bergey, John G. Holt (Editor), Noel R. Krieg, Peter H. A. Sneath, D. Bergy, Publisher: Lippincott, Williams & Wilkins). Gram negative bacteria includes, for example, bacteria of the following families: Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae et cetera.

Enterobacteriaceae family includes, for example, bacteria belonging to the genera Enterobacter, Erwinia, Escherichia, Klebsiella, Providencia, Salmonella, Serratia, Shigella et cetera.

The term “lacking active thymidylate synthase and thymidine kinase” means that the inherent genes coding for these enzymes are modified in such a way that the modified genes encode completely inactive proteins. It is also possible that the modified genes are unable to be expressed due to deletion of a part of the gene, shifting the reading frame, or through modification of adjacent region(s) of the genes, including sequences controlling operon expression, such as promoters, enhancers, attenuators etc.

It is known that cells which have lost thymidylate synthase activity cannot make DNA and cannot survive unless supplied with thymine or thymidine, which they convert to dTMP by an alternative pathway. Further inactivation of thymidine kinase can result in a bacterium which is unable to utilize thymine or thymidine present in the medium. As a result, the thymidylate synthase gene existing on the plasmid of the present invention became not only a selection marker, but also a factor for stabilization of the plasmid in the bacterium.

Thymidine kinase catalyzes ATP-dependent phosphorylation of thymidine yielding thymidine-5′-monophosphate (dTMP). The tdk gene which encodes thymidine kinase of Escherichia coli has been elucidated (nucleotide numbers 1292750 to 1293367 in the sequence of GenBank accession NC_{—}000913.1, gi:16129199). The tdk gene is located between the hns gene and ychG ORF on the chromosome of E. coli strain K12. The nucleotide sequence of tdk gene and the amino acid sequence encoded by the gene are shown in SEQ ID NOS: 46 and 47, respectively.

Inactivation of a gene can be performed by conventional methods, such as mutagenesis treatment using UV irradiation or nitrosoguanidine (N-methyl-N′-nitro-N-nitrosoguanidine) treatment, site-directed mutagenesis, gene disruption using homologous recombination or/and insertion-deletion mutagenesis (Datsenko K. A. and Wanner B. L., Proc. Natl. Acad. Sci. USA, 97:12: 6640-45 (2000)) also called as a “Red-driven integration”.

Particularly, inactivation of the host strain thyA gene is followed by transformation of the modified plasmid RSF1010 which has the mob locus and all the antibiotic resistance genes deleted and contains the thymidylate synthase gene (SEQ ID NO: 44) into a mutant host followed by further selection of transformants on a medium which does not contains thymidine. Then the inactivation of the tdk gene is performed. Inactivation of genes may be performed by substitution of the target gene with an antibiotic resistance gene flanked by sequences suitable for further excision of the antibiotic resistance gene. Systems for the excision are exemplified by the system using FRT sites and phage lambda Red recombinase (Flp recombinase) (Datsenko K. A. and Wanner B. L., Proc. Natl. Acad. Sci. USA, 97:12: 6640-45 (2000)), the system using attL and attR sites and products of int and xis genes from phage lambda (Peredelchuk, M. Y. and Bennett, G. N., Gene, 187, 231-238 (1997)), the system using loxP sites and Cre recombinase from bacteriophage P1 (Guo, F. et al, Nature, 389, 40-46), similar systems described by Campbell, A. M. (J. Bacteriol., 174, 23, 7495-7499 (1992)) and the like.

The bacterium of the present invention can be obtained by introduction of the plasmid of the present invention into a bacterium, whereby the bacterium has the inherent ability to produce a useful metabolite and lacks active thymidylate synthase and thymidine kinase. Alternatively, the bacterium of present invention can be obtained by imparting the ability to produce a useful metabolite to the bacterium already lacking active thymidylate synthase and thymidine kinase and harboring the plasmid.

The method of the present invention includes a method for producing a useful metabolite, comprising cultivating the bacterium of the present invention in a culture medium, allowing said metabolite to accumulate in the culture medium, and collecting the metabolite from the culture medium.

In the present invention, the cultivation, collection, and purification of target metabolite from the medium and the like may be performed in a manner similar to conventional fermentation methods wherein the target metabolite is produced using a microorganism. A medium useful for culture may be either synthetic or natural, so long as the medium includes a carbon source and a nitrogen source and minerals and, if necessary, appropriate amounts of nutrients which the microorganism requires for growth. The carbon source includes various carbohydrates such as glucose and sucrose, and various organic acids. Depending on the mode of assimilation of the chosen microorganism, alcohol, including ethanol and glycerol, may be used. As the nitrogen source, various ammonium salts such as ammonia and ammonium sulfate, other nitrogen compounds such as amines, a natural nitrogen source such as peptone, soybean-hydrolysate and digested fermentative microorganism may be used. As minerals, potassium monophosphate, magnesium sulfate, sodium chloride, ferrous sulfate, manganese sulfate, calcium chloride, and the like may be used. Additional nutrients, for example to complement auxotrophy, can be added to the medium, if necessary.

After cultivation, solids such as cells can be removed from the liquid medium by centrifugation or membrane filtration, and then the target metabolite can be collected and purified by conventional methods, such as ion-exchange, affinity chromatography, concentration, crystallization and other methods suitable for the specific desired metabolite.

### EXAMPLES

The present invention will be more concretely explained below with reference to the following non-limiting examples.

### Example 1

### Construction of the RSF1010mob⁻ Plasmid

Construction of the RSF1010 Mob⁻ plasmid was performed by “Red-driven integration” (Datsenko K. A. and Wanner B. L., Proc. Natl. Acad. Sci. USA, 2000, 97:12: 6640-45) of the DNA fragment containing an auto-regulated element P_{lacUV5}-lacI, marked by chloramphenicol resistance gene (cat gene) into the RSF1010 plasmid instead of the mob locus.

At first, a DNA fragment having a structural part of the lacI gene under control of the P_{lacUV5} promoter was amplified by PCR using primers P1 (SEQ ID NO: 29) and P2 (SEQ ID NO: 30) and the PMW-P_{lacUV5}-lacI-118 plasmid (Skorokhodova, A. Y. et al, Biotechnologiya (rus), No.5, (2004)) as a template. The nucleotide sequence of P_{lacUV5} promoter is disclosed in Genbank Accession No.Y00412 (nucleotides 7-100). The nucleotide sequence of lacI is disclosed in Genbank Accession No. NP_{—}414879. Furthermore, the nucleotide sequence of P_{lacUV5} promoter used in the present invention is described in SEQ ID NO: 24 (nucleotides 2824-2912). The P_{lacUV5} promoter can be obtained by chemical synthesis according to the nucleotide sequence of SEQ ID NO: 24, or by preparing from pET Expression System (Novagen). The nucleotide sequence of lacI is also described in SEQ ID NO: 25. The lacI can be obtained by PCR according to the nucleotide sequence of SEQ ID NO: 25 or GenBank Accession No. NP_{—}414879 using chromosomal DNA of E. coli K-12 (MG1655) as a template.

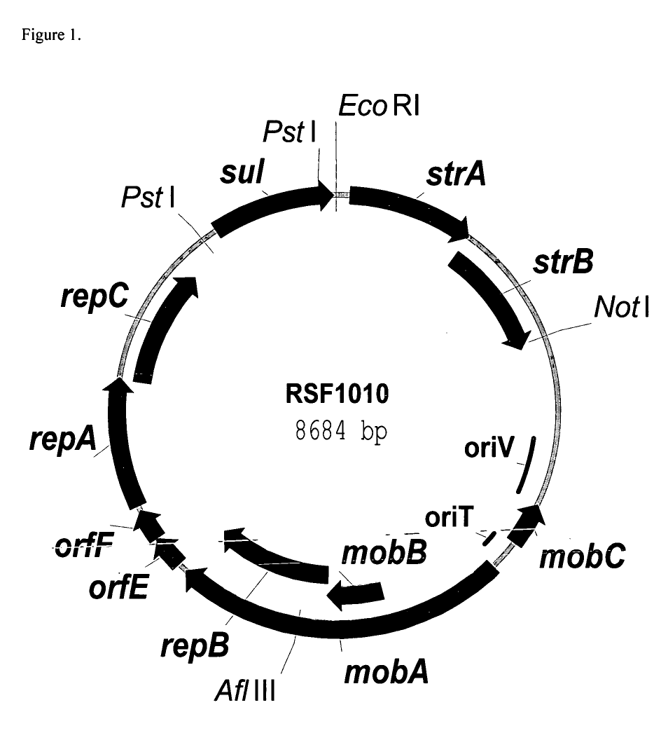

Primer P1 is identical to the region in the pMW-P_{lacUV5}-lacI-118 plasmid located upstream of the XbaI restriction site on the plasmid. Primer P2 contains a BamHI restriction site, which was introduced into the 5′-end thereof. A fragment of the repB (SEQ ID:13) gene from the plasmid RSF1010 was amplified by PCR using primers P3 (SEQ ID NO: 31) and P4 (SEQ ID NO: 32). The start codon of the repB gene and the stop codon of the mobB gene overlap on the plasmid RSF1010 (FIG. 1). The SD sequence of the repB gene is located 4 base pairs upstream of its start codon. To provide translation of RepB protein in the absence of the proximal mobB gene, the translation initiation region of the repB gene was modified by the addition of 4 nucleotides into the primer P3. Moreover, primer P3 contains a BamHI restriction site introduced into 5′-end thereof, and primer P4 contains a KpnI restriction site introduced into 5′-end thereof. The two obtained PCR products were purified by agarose gel electrophoresis, treated by BamHI restriction enzyme, ligated and used as a template for PCR using primers P1 and P4. The resulting DNA fragment was treated with XbaI and KpnI restrictases and cloned into pBluescript II SK(+) vector (Stratagene) which had been previously treated with the same restrictases. The resulting plasmid was named pBluescript::lacIrepB.

Then, a DNA fragment was constructed containing the chloramphenicol resistance gene (cat gene) and the P_{lacUV5} promoter. The cat gene was amplified from plasmid pACYC184 (Takara Bio) using primers P5 (SEQ ID NO: 33) and P6 (SEQ ID NO: 34). Primer P5 contains a BglII restriction site, which was introduced into 5′-end thereof and is necessary for further excision of the cat gene after selection of the mob⁻ plasmid. Primer P6 contains a SacI restriction site introduced into 5′-end thereof. The P_{lacUV5} promoter was amplified from a PMW-P_{lacUV5}-lacI-118 plasmid using primers P7 (SEQ ID NO: 35) and P8 (SEQ ID NO: 36). Primer P7 contains a SacI restriction site introduced into 5′-end thereof. Primer P8 contains an XbaI restriction site introduced into 5′-end thereof. The obtained fragments were purified, by agarose gel electrophoresis, treated with SacI restrictase, ligated and used as a template for PCR using primers P5 and P8. Then, the resulted product was treated with XbaI restrictase and ligated with the pBluescript::lacIrepB plasmid which had been previously treated with the same restrictase. The obtained linear product was used as a template for PCR with primers P4 (SEQ ID NO: 32) and P9 (SEQ ID NO: 37). Primer P9 contains 38 nucleotides of the RSF1010 region, which is located between the oriV and the 3′-end of the mobC gene, BglII restriction site and 17 nucleotides complementary to the 5′-end of cat gene.

The obtained PCR product, which contains the 3′-end of repB gene, the lacI gene under control of the P_{lacUV5} promoter, the cat gene and the 38 nucleotides of the RSF1010 region located between the oriV and the 3′-end of mobC gene, was used for the integration into the RSF1010 plasmid, substituting for the mob locus of the plasmid, using “Red-driven integration” (Datsenko K. A. and Wanner B. L., Proc. Natl. Acad. Sci. USA, 2000, 97:12: 6640-45). According to the procedure, plasmid pKD46 was used as a helper plasmid. Escherichia coli strain BW25113 containing the recombinant plasmid pKD46 can be obtained from the E. coli Genetic Stock Center, Yale University, New Haven, USA, the accession number of which is CGSC7630.

The RSF1010 plasmid was introduced into the strain MG1655(pKD46) together with DNA fragment described above by electroporation. The strain MG1655 (ATCC No. 47076) is available from the American Type Culture Collection (ATCC, Address: P.O. Box 1549, Manassas, Va. 20108, United States of America).

100-200 ng of the PCR-amplified DNA fragment and 100 ng of the RSF1010 plasmid were used for electroporation. Electroporation was performed using electroporator BioRad (No. 165-2098, ver.2-89, USA) (impulse time was 4-5 msec, electric field strength was 12,.5 kV/cm). After electroporation 1 ml of SOC medium was immediately added to the cell suspension. The cells were grown at 37° C. for 2 hours, and were spread on LB agar containing 30 μg/ml of chloramphenicol and then were grown at 37° C. overnight.

The isolated RSFmob⁻ cat plasmid obtained as a result of the homologous recombination was treated with BglII and XbaI restrictases to remove the cat gene and then was ligated with the PCR fragment containing P_{lacUV5} promoter treated with the same restrictases. A PCR fragment containing the P_{lacUV5} promoter was obtained using primers P1 (SEQ ID NO: 29) and P8 (SEQ ID NO: 36). The sequence of the RSF1010 derivative with a deleted mob locus (RSF1010mob⁻, 8338 bp) is presented in the Sequence Listing in SEQ ID NO: 24.

As for the stability of the obtained plasmid, seven passages of the plasmid-carrier culture were provided in non-selective conditions and among 100 independent clones, no streptomycin sensitive (Sm^{S}) clones were obtained. Therefore, the stability of the obtained plasmid RSF1010mob⁻ was not lower than 99% after seven passages without selection.

The mobilization efficiency of RSF1010mob⁻ plasmid as compared to the parental plasmid RSF1010 was investigated. For this purpose, the donor strain was constructed on the basis of E. coli strain C600 (r+m+)(Funakoshi), which contains resident plasmid RP1-2 (Tc^{r}). This plasmid provides the tra-operon genes necessary for conjugative transfer. Plasmids RSF1010 and RSF1010mob⁻ were introduced by transformation into C600 (RP1-2) strain using Str^{r} as a selective marker. Three control donor strains were constructed by transformation with plasmids pAYC32 (Chistoserdov, A. Y. and Tsygankov, Y. D., Plasmid, 16, 161-167 (1986)), pBR322 and pUC19. All of the constructed donor strains were used in conjugation experiments with the recipient strain LE392 met⁻ Rif^{R} (Promega) to determine the mobilization efficiency. The results of these experiments are presented in Table 2.

TABLE 2

| Plasmid in donor with RP1-2 | Mobilization frequency^{a} |
|---|---|
| RSF1010^{b} | 2.5 × 10⁻⁵ |
| RSF1010mob⁻ | <10⁻⁸ |
| pAYC32^{c} | 3 × 10⁻⁴ |
| pBR322^{c} | 4 × 10⁻⁶ |
| pUC19^{c} | <10⁻⁸ |

^{a}Number of transconjugants per donor after overnight mating of C600 donor and LE392 as the recipient.
^{b}The mobilization of RSF1010 and its derivatives was determined on LB-rifampicin-streptomycin medium.
^{c}The mobilization of plasmids were determined on LB-rifampicin-ampicillin medium.

The results of Table 2 indicate that plasmid RSF1010mob⁻ completely lost mobilization ability. In this respect, they are similar to the pUC19 control plasmid which doesn't contain any mob genes.

### Example 2

### Construction of the mob⁻ Derivative of RSF1010 Plasmid having Increased Copy Number

According to our data, in the stationary phase the RSF1010mob⁻ had the same copy number as RSF1010. In logarithmic growth phase the copy number of the obtained derivative was about two times lower than the copy number of RSF1010 plasmid. The addition of IPTG to cultivation medium caused an increase of the copy number of RSF1010mob⁻ plasmid in the logarithmic growth phase because repB gene involved in replication of RSF-like plasmids is placed under transcriptional control of P_{lacUV5}-lacI auto-regulated element. So, it was proposed that elimination of the lacI gene from RSF1010mob⁻ plasmid could increase the copy number of the plasmid. Corresponding derivative of RSF1010mob⁻ without the lacI gene was obtained by excision of lacI gene from RSF1010mob⁻ plasmid using XbaI and BamHI restrictases. Then sticky ends of resulting DNA fragment were blunted and RSF1010mob⁻, lacI⁻ plasmid was obtained by ligation. The DNA sequence of RSF1010mob⁻, lacI⁻ plasmid was shown in SEQ ID NO: 48.

To estimate the copy numbers of derivatives of RSF1010 plasmid, three plasmids were separately introduced into E. coli strain MG1655. Plasmid DNA had been isolated from equal quantities of cells grown overnight in LB medium without IPTG using “GenElute Plasmid Miniprep Kit” (Sigma, USA), treated with EcoRV restrictase and RNAse A. The copy numbers of plas-r-ds were estimated using “Sorbfil” program by scanning of electrophoretic bends corresponding to the large EcoRV fragments of each plasmid after coloration of the agarose gel with ethidium bromide. Three independent transformants of each type have been used in this experiment. The relative copy numbers of derivatives of RSF1010 are presented in Table 3. Copy number of RFS1010 plasmid was taken as 1.0.

TABLE 3
Relative copy number of the mob⁻ derivatives of RSF1010 plasmid.

| Plasmid | Relative copy number |
|---|---|
| RSF1010 | 1,0 ± 0,3 |
| RSF1010mob⁻ | 0,9 ± 0,1 |
| RSF1010mob⁻, lacI⁻ | 2,6 ± 0,3 |

### Example 3

### Construction of the RSF1010 Mob⁻ Plasmid Lacking any Antibiotic Resistance Genes and Containing the thyA Gene as a Selection Marker

At first, two strains were constructed on the basis of wild-type E. coli strain MG1655; one strain had a thyA gene deleted, and the other had a tdk gene deleted. So called “Red-driven integration” method (Datsenko K. A. and Wanner B. L., Proc. Natl. Acad. Sci. USA, 2000, 97:12: 6640-45” was used for inactivation of target genes by integration of the fragment comprising antibiotic-resistance markers into each of the above strains. Chloramphenicol resistance gene from plasmid pACYC184 was used for disruption of thyA gene (Cm^{r}) and kanamycin resistance gene from plasmid pACYC177 was used for disruption of tdk gene (Km^{r}). Both of the obtained mutants carrying the antibiotic resistance marker may be used as donors for P1 transduction of ΔthyA and Δtdk deletions into other E. coli strains.

The strain with the thyA deletion was used in the present invention for screening of functionally active copy of thyA gene cloned on different plasmids.

The second stage included cloning of a functionally active copy of thyA gene. In the chromosome, of E. coli MG1655 strain, the thyA gene is located in a distal part of proposed operon structure—lgt-thyA. There could be a proximal promoter for this operon. Although thyA gene possesses two annotated promoters just near the start codon (P_{thyA1} and P_{thyA2}), their sequences differ from the canonical one, so their potential respective efficiencies were under question.

In accordance with the physical map of the E. coli chromosome, the structural thyA gene consists of 795 bp and the corresponding protein thymidylate synthase contains 264 amino acids. The nucleotide sequence of thyA gene and the amino acid sequence encoded by the gene are shown in SEQ ID NOS: 44 and 45, respectively.

The structural part of the thyA gene containing both native promoters was amplified by PCR using primers ThyA1 (SEQ ID NO: 38) and ThyA2 (SEQ ID NO: 39) and chromosomal DNA from E. coli cells TG1 (Amersham Pharmacia Biotech). These primers contain EcoRI and HindIII restriction sites, respectively, for cloning of thyA gene into vectors pUC18 (Takara Bio), pUC19 (Takara Bio) and pET22(+) (Promega). After PCR amplification, a 994 bp fragment of DNA containing the thyA gene was isolated and cloned into EcoRI and HindIII sites of plasmids pUC18, pUC19 and pET22(+). After transformation into E. coli strain TG1, Amp^{R} clones were isolated and tested in the presence of the cloned thyA gene using control PCR with primers ThyA1 and ThyA2. Several clones containing the expected DNA fragments were chosen for isolation of recombinant plasmids and determination of functional activity of cloned thyA gene.

To determine the functional activity of the thyA gene cloned on plasmids pUC18, pUC19 and pET22(+) all plasmids were introduced by transformation into recipient cells of strain MG1655(ΔthyA::Cm^{r}). 50 independent Amp^{R} clones from each transformation experiment were selected and checked for the ability to complement thyA mutation. The presence of cloned thyA gene in clones containing plasmids pUC18 thyA, pUC19 thyA and pET22(+) thyA was confirmed by control PCR with ThyA1 and ThyA2 primers. Furthermore, it was shown that all the transformants tested, except those derived from plasmid pET22, were able to grow on minimal medium without thymidine. These data indicate that the cloned thyA gene is able to express from its own promoter only under conditions when it was cloned on multicopy plasmids PUC18 and pUC19.

It should be noted that the EcoRI-HindIII fragment containing the thyA gene cloned on pUC18 and pUC19 plasmids is in opposite orientation. Thus, the pUC18 plasmid transcription of thyA gene coincides with the transcription of plasmid lacZ gene, i.e. transcription of thyA gene may occur from lacZ promoter, while in pUC19 plasmid transcription of thyA gene may be directed only from its own promoter. Therefore, a comparison of expression of thyA cloned on pUC18 and pUC19 plasmids allow one to estimate the efficacy of the thyA promoter. It was found that clones of MG1655(ΔthyA::Cm^{r}) strain containing pUC18 thyA plasmids grow better on minimal medium as compared to clones containing pUC19 thyA plasmids. These data allow us to suggest that the level of thyA transcription from its own promoter is lower when compared to a construction containing the lacZ promoter upstream.

On the other hand, recipient strain MG1655(ΔthyA::Cm^{r}) containing pET22(+) vector with cloned thyA gene can grow very slow on minimal medium without thymidine. This data indicate that expression of thyA gene from its own promoter on plasmid pET22(+) is not enough to support growth of the recipient strain MG1655(ΔthyA::Cm^{r}). It is known that the copy number of the pET22 plasmid is lower as compared to pUC18 (19) plasmids. Because our final vector RSF1010 is also not a very high copy number plasmid, it was decided to improve expression of thyA gene cloned on pET22(+) vector. For that purpose some additional mutations into −10 region of thyA gene promoter were introduced using site-directed PCR mutagenesis.

Two primers were designed: ThyA4 (SEQ ID NO: 40) and ThyA5 (SEQ ID NO: 41). Both primers contain substitutions in the −10 region of the promoter and also in the TG motif at positions −15 and −14, which must improve the efficiency of transcription from the thyA promoter. Two separate PCR amplifications with pairs of primers ThyA1-ThyA5 and ThyA2-ThyA4 and pET-22-thyA plasmid as a template were conducted, and two fragments of thyA gene were isolated. Then, the products of the PCR amplification were annealed together and the resulting mixture was used as a template for PCR with ThyA1 and ThyA2 primers to isolate the full size thyA gene with the improved −10 region. After PCR amplification, this modified 994 bp fragment was digested with EcoRI and HindIII restrictases and cloned into vectors pUC18, pUC19 and pET22 which had been previously treated with the same restrictases. After transformation into E. coli strain TG1, Amp^{R} clones were isolated and tested in the presence of the cloned thyA gene by using control PCR with ThyA1 and ThyA2 primers. Several clones containing the expected DNA fragments were chosen for isolation of recombinant plasmids, sequencing and determination of functional activity of cloned thyA gene.

First of all, the modified thyA gene (hereinafter designated as thyA* gene) was sequenced and the presence of the introduced mutation in the promoter region was confirmed. The new promoter contains a perfect Pribnow-box: TATAAT and also the TG motif at positions −15 and −14, respectively (FIG. 1 nucleotides 87-95 in SEQ ID:27).

To check the ability of the improved thyA promoter to provide the level of thyA* gene expression sufficient for growth of thyA auxotrophs, plasmids pUC18, pUC19 and pET22(+) containing thyA* gene under control of the modified promoter were transformed into recipient cells of strain MG1655(ΔthyA::Cm^{r}). 50 independent Amp^{R} clones from each transformation experiment were selected and checked for their ability to complement thyA mutation. The presence of cloned thyA* gene in clones containing plasmids pUC18 thyA*, pUC19 thyA* and pET22 thyA* was confirmed by control PCR with ThyA1 and ThyA2 primers. Furthermore, it was shown that all transformants tested, including those containing the pET22 plasmid, were able to grow on minimal medium without thymidine. These data indicate that the activity of thymidylate synthase under the control of improved thyA* promoter is sufficient for growth of thyA auxotrophs.

One more modification of the thyA* gene was required to get rid of PstI restriction site in the structural part of the gene. This site was planned for cutting the Sul^{R} gene(SEQ ID:22) from the RSF1010mob- plasmid. The modification of the structure of functionally active gene was undertaken using the PCR technique as described above for site-specific mutagenesis to modify the promoter region. Using two separate PCR amplifications with pairs of primers ThyA1-ThyA16 and ThyA17-ThyA2 and pET-22-thyA* plasmid as a template, two fragments of thyA gene were isolated. Primers ThyA16 (SEQ ID NO: 42) ThyA17 (SEQ ID NO: 43) provided an introduction of a synonymic codon eliminating the PstI restriction site from the structural part of thyA gene. Then the products of PCR amplification were annealed together and resulted mixture was used as template for PCR with ThyA1 and ThyA2 primers to isolate full size thyA* gene without the PstI restriction site in its structural part. After PCR amplification, this modified 994 bp fragment was digested with EcoRI and HindIII restrictases and cloned into vectors pUC18, pUC19 and pET22 previously treated with the same restrictases. After transformation into E. coli strain TG1, AmP^{R} clones were isolated and tested on the presence of cloned thyA* gene by using control PCR with ThyA1 and ThyA2 primers. Several clones containing the expected DNA fragments were chosen for isolation of recombinant plasmids, sequencing and determination of functional activity of cloned thyA* gene.

### Example 4

### Substitution of Antibiotic Resistance Markers (Str^{R} and Sul^{R}) of RSF1010mob- with the thyA* Gene

One of clones isolated was used for isolation of plasmid pET22(+) containing a modified thyA* gene. Plasmid DNA was digested by EcoRI and NotI restrictases for subcloning into corresponding sites of the RSF1010mob- plasmid. The ligase mixture was transformed into the recipient strain MG1655(ΔthyA::Cmr) and ThyA⁺ transformants were isolated on minimal glucose medium without thymidine. ThyA⁺ transformants were checked for the presence of thyA* gene within recombinant RSF1010 plasmid by using PCR with primers ThyA1 and ThyA2 flanking thyA* gene. It was shown that all ThyA⁺ transformants tested exhibited sensitivity to streptomycin. These data indicate that the new isolated vector RSF1010mob- thyA* (without PstI site) contains substitution of Str^{R} gene encoded by strA and strB gene (SEQ ID NO:2 and 4) by thyA* gene. For the next step, plasmid RSF1010mob- thyA* was digested by PstI restrictase and self-ligated in order to delete Sul^{R} marker encoded by sul gene(SEQ ID NO:22). As a result, the new RSF1010mob- thyA* plasmid with substitution of both (Str^{R} and Sul^{R}) antibiotic resistance markers by thyA* selective marker was isolated. The sequence of the derivative of RSF1010 having mob locus and all antibiotic resistance genes deleted and containing thymidylate synthase gene (thyA* gene) as a selection marker is presented in the Sequence Listing in SEQ ID NO: 27. The new plasmid was named RSF1010-MT.

### Example 5

### Investigation of the Stability of the RSF1010-MT Plasmid in the thyA⁻, tdk⁻ Recipients

Strain MG1655(ΔthyA::Cm^{r}) was transformed with RSF1010-MT plasmid and ThyA⁺ transformants were isolated on minimal medium without thymidine. In accordance with the physical map of the E. coli chromosome, the structural tdk gene consists of 618 bp and the corresponding protein thymidylate synthase contains 205 amino acids. (SEQ ID NOS: 46 and 47).

Then, we performed P1 transduction experiments with phage P1 stock grown on MG1655 strain containing tdk::Km^{R} insertion on the chromosome (MG1655(Δtdk::Km^{r})) and recipient strain MG1655(ΔthyA::Cm^{r}) carrying RSF1010-MT plasmid. Kanamycin-resistant colonies were obtained and checked by PCR for the presence of tdk::Km^{R} insertion on the chromosome.

After isolation of MG1655(ΔthyA::Cm^{r}, Δtdk::Km^{r})/RSF1010-MT strain, investigation of stability of RSF1010-MT plasmid during propagation under nonselective conditions was performed. For this purpose recipient cells of MG1655(ΔthyA::Cm^{r}, Δtdk::Km^{r})/RSF1010-MT were grown in LB-broth in tubes at 37° C. for 72 hours. Then, samples of the culture were spread on LB-plates and single colonies which appeared after 24 h were replicated (200 colonies for each culture) on minimal medium without thymidine. The results indicated that all 200 colonies derived from MG1655(ΔthyA::Cm^{r}, Δtdk::Km^{r}) strain containing RSF1010-MT plasmid can grow on minimal medium without thymidine, i.e. all recombinants tested exhibited stable maintenance of vectors.

These data indicate that thyA* gene cloned on RSF1010mob⁻ plasmid provides stable maintenance of the plasmid as a selective marker instead of antibiotic resistance markers.

While the invention has been described in detail with reference to preferred embodiments thereof, it will be apparent to one skilled in the art that various changes can be made, and equivalents employed, without departing from the scope of the invention. All the cited references, including Russian patent application 2004119027, herein are incorporated as a part of this application by reference.

We claim:

1. A RSF1010 derivative Mob⁻ plasmid, wherein said plasmid is selected from the group consisting of SEQ ID NO: 24, SEQ ID NO: 27 and SEQ ID NO: 48 and variants of SEQ ID NO: 24, SEQ ID NO: 27 and SEQ ID NO: 48 which are at least 95% homologous to SEQ ID NO: 24, SEQ ID NO: 27 and SEQ ID NO: 48 and wherein said plasmid has been modified to inactivate a gene or genes related to mobilization ability.

2. The plasmid according to claim 1, wherein the plasmid has been modified to inactivate an antibiotic resistance gene.

3. The plasmid according to claim 1, wherein the plasmid has been modified to increase the copy number of the plasmid.

4. The plasmid according to claim 1 comprising a PlacUV5 promoter and an origin of replication from RSF1010 without a mob locus.

5. The plasmid according to claim 1, additionally comprising a thymidylate synthase gene.

6. The plasmid according to claim 1, additionally comprising a gene of interest.

7. A bacterium comprising the plasmid of claim 1.

8. The bacterium according to claim 7, wherein said bacterium is a Gram negative bacterium.

9. The bacterium according to claim 8, wherein said bacterium lacks active thymidylate synthase and lacks active thymidine kinase.

10. The bacterium according to claim 9, wherein said bacterium has an ability to produce a useful metabolite.

11. The bacterium according to claim 10, wherein said useful metabolite is selected from the group consisting of native or recombinant proteins, enzymes, L-amino acids, nucleosides, nucleotides, organic acids and vitamins.

12. A method for producing a useful metabolite, comprising
(a) cultivating the bacterium according to claim 10 in a culture medium and
(b) collecting said useful metabolite from the culture medium.

13. The method according to claim 12, wherein said useful metabolite is selected from the group consisting of native or recombinant proteins, enzymes, L-amino acids, nucleosides, nucleotides, and vitamins.
